(19) United States
(12) Patent Application Publication
(10) Pub. No.: US 2004/0072246 A1
(43) Pub. Date: Apr. 15, 2004

(54) System and method for identifying t cell and other epitopes and the like

(76) Inventors: Roland Martin, Bethesda, MD (US); Richard Simon, Chevy Chase, MD (US); Yingdong Zhao, Rockville, MD (US); Bruno Gran, Cynwyd, PA (US); Clemencia Pinilla, Cardiff by the Sea, CA (US)

Correspondence Address: NATIONAL INSTITUTES OF HEALTH, OFFICE OF TECHNOLOGY TRANSFER, 6011 EXECUTIVE BLVD SUITE 325, ROCKVILLE, MD, 20852-3804, US

(21) Appl. No.: 10/380,147

(22) PCT Filed: Sep. 11, 2001
(86) PCT No.: PCT/US01/42166

(51) Int. Cl. G01N 33/53; G06F 19/00; G01N 33/48; G01N 33/50

(52) U.S. Cl. 435/7.1; 702/19

(57) ABSTRACT

A system and method is described for identifying T cell and other epitopes and the like.

### FIELD OF THE INVENTION

A system and method is described for identifying T cell and other epitopes and the like.

### BACKGROUND OF THE INVENTION

CD8⁺ and CD4⁺ T lymphocytes recognize short peptides of 8-10 and 12-16 amino acids in the context of self MHC-class I and -class II molecules respectively (Cresswell P., 1994, Annu Rev Immunol 12 259-93; Engelhard V. H., 1994, Annu Rev Immunol 12: 181-207). During the last 15 years, this central process of cellular immune responses has received enormous attention and has been dissected using a vast array of different immunological and biochemical techniques. A quantitative analysis of the interaction between T cell receptors (TCR) and their MHC-peptide ligands would be an important basis for the design of vaccines and therapeutic approaches to immune-mediated, infectious and neoplastic diseases.

Because it has been difficult to describe the trimolecular complex in its entirety, experiments initially focused on the interaction between peptide and MHC molecules. Structural studies of MHC-class I and -class II molecules complexed with antigenic peptides disclosed that the latter bind in a linear fashion (Madden D. R. 1995, Annu Rev Immunol 13: 587-622). Sequencing of peptide pools and of individual self peptides eluted from MHC molecules (Falk K. et al, 1994, Immunogenetics 39 230-42; Verreck F. X et al., 1994, Eur J Immunol 24: 375-9) together with systematic binding analyses (Rothbard J. B., and Gefter M. L., 1991 Annu Rev Immunol 9: 527-565; Sette A. et al., 1994, Mol Immunol 31: 813-22) have provided experimental data for the definition of MHC-binding motifs (Hammer J. et al., 1994, J Exp Med 180; Hammer J. et al., 1993, Cell 74: 197-203; Rammensee, H. G. et al., 1995 Immunogenetics 41: 178-228; Sette A. et al., 1989, PNAS USA 86: 3296-300; Sturniolo T. et al., 1999, Nat Biotechnol 17: 555-561) and the development of MHC-peptide binding models. A combination of positive and negative influences from amino acid side chains in the antigenic peptide has been shown to determine the interaction between peptide and MHC molecules (Hammer J., 1995, Curr Opin Immunol 7: 263-9). Indeed, the assumption of independent contribution of each amino acid side chain in the peptide sequence to MHC binding has been used to develop quantitative methods that predict peptide binding to MHC alleles (Hammer J. et al, 1994, J Exp Med 180; Mallios, R. R. 1994, J Theor Biol 166: 167-72; Parker K. C. et al., 1994, J Immunol 152: 163-75; Southwood S. et al., 1998, J Immunol 160: 3363-73). More recently, elegant neural network approaches have been used to further refine the prediction of peptide binding to MHC (Brusic V. et al., 1998, Bioinformatics 14: 121-30; Gulukota K. et al., 1997, J Mol Biol 267: 1258-67; Honeyman M. C. et al., 1998, Nat Biotechnol 16:966; Milik M. et al., 1998, Nat Biotechnol 16:753). Based on the fact that a subset of MHC-binding peptides are also T-cell epitopes (Davenport M. P. et al., 1995, Immunogenetics 42:392; Roberts C. G. et al., 1996, AIDS Res Hum Retroviruses 12:593), MHC-binding has been used to predict candidate T-cell epitopes in bulk T-cell populations, such as those contained in the peripheral blood (Sturniolo T. et al., 1999, Nat Biotechnol 17:555; Honeyman M. C. et al., 1998, Nat Biotechnol 16:966). However, to dissect and predict precisely the interaction of all three components of the trimolecular complex has until now been a difficult undertaking. The quantitative study of MHC-peptide recognition by single TCR has therefore remained a largely unsettled issue.

The specificity of the trimolecular complex interaction has been studied using individual substitution analogs. Although initial studies showed that some amino acids in the antigenic peptide sequence are necessary for recognition by the TCR (primary TCR contacts) and others can tolerate conservative substitutions (secondary contacts) (Kersh G. J., P. M. Allen. 1996, J Exp Med 184:1259; Sloan-Lancaster, J., P. M. Allen. 1996, Annu Rev Immunol 14:1), the systematic use of single- and multiple amino acid-substituted peptides has shown that all amino acid side chains can contribute to peptide recognition in a largely independent manner (Hemmer B. et al., 1998, J Immunol 160:3631). In extreme cases, this can lead to recognition of peptides with entirely different amino acid sequences by the same TCR (Hemmer B. et al., 1998, J Immunol 160:3631).

The development of soluble- and bead-bound combinatorial peptide libraries in various formats representing millions to trillions of peptides has emerged as a powerful approach to both T-cell epitope determination and the analysis of TCR specificity and flexibility as recently reviewed (Piillla C. et al., 1999, Curr Opin Immunol 11:193; Hiemstra H. S. et al., 2000, Curr Opin Immunol 12:80). Recent studies (Gundlach B. R. et al., 1996, J Immunol 156:3645; Gundlach, B. R. et al., 1996, J Immunol Methods 192:149; Udaka K. et al., 1996, J Immunol 157:670; Wilson D. B. et al., 1999, J Immunol 163:6424; Hemmer B. et al., 2000, J Immunol 164:861) of T cell clones demonstrated the efficacy of using positional scanning synthetic combinatorial libraries (PS-SCL) for identifying target antigens and highly active peptide mimics. It was, however, technically impossible to fully utilize this technology without the development of quantitative methods for predicting the stimulatory potential of peptides based on data from these complex libraries.

### SUMMARY OF THE INVENTION

We report here systems and methods that combine data acquisition with positional scanning synthetic combinatorial libraries (PS-SCL) and analysis with a quantitative scoring matrix in order to identify agonist peptides for clonotypic T cell receptors (TCR) of known and unknown specificity. Peptides can be identified from database searches with unprecedented efficiency and ranked according to a score that is predictive of their stimulatory potency. To our knowledge, this is by far the most efficient available approach to identify stimulatory peptides for individual TCR and predict their actual stimulatory potency with relatively high accuracy. By this prediction strategy, we have developed a tool for the identification of potential T-cell epitopes, the design of vaccines, and the quantitative analysis of TCR degeneracy. Finally, we demonstrate how the invention can be similarly employed to identify the interacting partners of any receptor-ligand interaction, e.g., opioid with opioid receptors.

### BRIEF DESCRIPTION OF THE DRAWINGS

FIG. 1. Proliferative response of T-cell clones GP5F11 (A) and TL3A6 (B) to the 200 mixtures of a decapeptide PS-SCL in which each position has one defined amino acid (20 for each of the 10 positions; the single letter amino acid code is used). Proliferation is shown as c.p.m. induced by each mixture of the PS-SCL (mean and standard deviations of duplicate wells). *, Proliferation in the absence of peptide mixtures. TCC GP5F11 is specific for an influenza virus hemagglutinin (HA)-derived peptide, Flu-HA₃₀₈₋₃₁₇; TCC TL3A6 is specific for a myelin basic protein (MBP)-derived peptide. The corresponding sequences of HA₃₀₈-317, YVKQNTKLA (SEQ ID NO:1), and MBP₈₉₋₉₈, FFKNIVTPRT (SEQ ID NO:2), are indicated by diamonds at the top of each panel. Proliferation in the absence of antigen was 124±42 c.p.m. (panel A) and 1453+493 c.p.m. (panel B).

FIG. 2. Flow diagram of the strategy used to quantitatively analyze TCR recognition of antigens by clonotypic T cells. Experimental data collected by measuring functional T-cell responses to PS-SCL are then analyzed by a scoring matrix approach. This allows the identification and ranking of the spectrum of antigenic ligands for TCC of known and unknown specificity.

FIG. 3. A Score matrix for TCC GP5F11. Data from a representative experiment of proliferative response of the TCC to a decamer PS-SCL experiment are used to generate the matrix. Each number represents the S-index (c.p.m. in the presence of the mixture/c.p.m. in the absence of the mixture) of each of the 200 mixtures of a decapeptide PS-SCL (20 amino acids, indicated by the single-letter code, for each of the 10 positions of a decamer peptide, P1 to P10). In a model of independent contribution of each amino acid to peptide recognition, the stimulatory value of any decapeptide can be determined by summing the values of the individual amino acids in the score matrix. The example shown is a decamer peptide derived from influenza virus hemagglutinin HA₃₀₈₋₃₁₇ that was used to establish the TCC. Boxed numbers correspond to the amino acid sequence of the peptide and their sum represents the peptide score. Also shown are the maximum and minimum scores that can be assigned to any decamer peptides by this particular matrix. B, The scoring matrix can be used to score contiguous decamer peptides contained in all known protein sequences contained in public databases in order to find stimulatory peptides for a given T-cell clone. The example shows a decamer scoring “window” moved in one amino acid increments along the sequence of influenza virus hemagglutinin (HA), recognized by TCC GP5F11. The matrix (FIG. 3) derived from a representative PS-SCL experiment (FIG. 1A) attributes the highest score to a decamer peptide (308-317) corresponding to the core of the 13-mer used to establish the TCC (HA₃₀₆₋₃₁₈). Dramatic changes can be shown by scoring the overlapping decamer peptides along the entire sequence (panel B). Remarkably, the highest score correspond to the actual epitope recognized by the TCC.

FIG. 4. Proliferative response of the TCC GP5F11 to representative agonist peptides identified by the peptide library strategy. The potency is highest for a theoretical peptide that is predicted to be a potent one because it has a high score. The native peptide (influenza virus HA₃₀₈₋₃₁₇) and a double-substituted naturally occurring variant have intermediate potency. A low-scoring peptide derived from H. sapiens phosphatidylinositol-4-phosphate 5-kinase type III (PIP5 KIII₂₄₆₋₂₅₅) and a theoretical peptide predicted to be non-stimulatory because it has a very low score are indeed non-stimulatory.

FIG. 5. A, Upregulation of Titin gene expression in lesions of two MS patients. Levels of Titin expression in individual lesions from two MS patients (R and W). Bars represent ratios of expression of Titin in the indicated 18 lesions relative to Titin expression in pooled normal white matter. B, Identification of a potential autoantigen expressed in MS lesions by the integrated approach of peptide combinatorial libraries and cDNA microarray analysis. Two T-cell clones reactive to myelin and microbial antigens were analyzed for their pattern of antigen recognition by the PS-SCL approach and a numeric matrix was used to score and rank predicted stimulatory peptides for their potency (left). Gene expression in MS lesions and normal white matter was compared by cDNA microarray analysis and a number of overexpressed genes was identified (right). The comparison of predicted stimulatory peptides and overexpressed genes identified interesting candidate target autoantigens such as the giant protein Titin. C, Proliferative response of TCC CSF-3 to a Titin-derived peptide. TCC CSF-3 was isolated from the cerebrospinal fluid of a patient with chronic neuroborreliosis and recognizes a lysate of B. burgdorferi as well as a number of peptides derived from B. burgdorferi, human self antigens, and viral antigens. The proliferative response (in c.p.m.) to Titin (6205-6214) (GenBank accession No X90569) is shown in one representative experiment. The background (no antigen) control proliferation was 198 c.p.m.

FIG. 6: T-cell clonotypes of umnanipulated CSF, examined by RT-PCR-single-strand conformation polymorphism. a, Each distinct band indicates accumulation of a single T-cell clone. Dominant T-cell clonotypes express TCR V, 5.1, 5.2, 6, 7, 8, 13.2, 14 and 18 (underlined). One of the TCR Vβ 14-bearing clonotypes (arrow) corresponds to CSF-3. b, T-cell clone (TCC)CSF-3 corresponds to one of the TCR VP clonotypes of freshly isolated CSF T cells. c, The TCR Vβ junctional sequence of Tell clone CSF-3: the last eight amino acids of the variable (Vβ 14) segment, followed by the junctional sequence (n-D-n and TCR Jβ 2.3) and the first four residues of the constant region (TCR Cβ) of the T-cell clone.

FIG. 7. Proliferative response of T-cell clone CSF-3 to the 200 mixtures of a decapeptide PS-SCL in which each position has one defined amino acid (20 for each of the 10 positions (P1 to P10); horizontal axes, single-letter amino acid code). Vertical axes, proliferation, as counts per minute (c.p.m.) induced by each mixture of the PS-SCL (mean and standard deviations of duplicate wells). Data represent one experiment of five.

FIG. 8. Score distributions for sequences for all putative peptides 10 amino acids in length. Scores were generated by using the score matrix of the T-cell clone CSF-3 to ‘slide’ over the whole genomes of Borrelia burgdorferi (⋄), Treponema pallidum (∘), Mycobacterium tuberculosis (□) and Escherichia coli (Δ). Vertical axis, percentage of the amount of 10-amino-acid peptides in each organism; horizontal axis, score range, calculated by: [score−min(score)]/[max(score)−min(score)]×100, where min(score) and max(score) are the minimum and maximum scores, respectively, that can be generated from the scoring matrix Vertical dotted line, 60% of the maximum score; inset, tails of the distribution curves beyond the 60% cut-off.

FIG. 9. Activation of T-cell clone CSF-3 by the identified peptides. a, Proliferative response of the T-cell clone CSF-3 to representative agonist peptides identified by the peptide library strategy. There is a spectrum of potency for the response to peptides derived from B. burgdorferi (peptides 26 (●), 37 (∘) and 54 (▾) and Homo sapiens (peptide 62 (∇); Table 9). Right, response to serial dilutions (in volume/volume) of B. burgdorferi lysate in the same experiment. b, HLA restriction of the response to peptides 59 (derived from B. burgdorferi) and 71 (from human aminopeptidase A; Table 9). There is a proliferative response to both the peptides (10 ng/ml) and a 1:100 dilution (volume/volume) of B. burgdorferi lysate (B.Lys 1/100) when DR2b-transfected (right), but not DR2a-transfected (left), bare lymphocyte syndrome cells are used as antigen-presenting cells. c, Effect of peptides 59 and 71 on tyrosine phophorylation of TCR subunits. CSF-3 cells were stimulated with bare lymphocyte syndrome cells alone or each peptide for 5 or 10 min (time, below blot). TCR subunits were immunoprecipitated from cell lysates using rabbit antiserum against ZAP-70, then immunoprecipitates were immunoblotted using a monoclonal antibody against phosphotyrosine (4G10). Right margin, TCRζ-chain phosphoisoforms p38 and p32.

FIG. 10. Represents the scoring distribution of all the hexapeptides in the human database in biometrical analysis for opioid receptors.

FIG. 11 is a block diagram illustrating one embodiment of a system for determining binding epitopes of a T cell.

FIG. 12 is a flow diagram illustrating one embodiment of a process for determining proteins that bind to a T cell.

FIGS. 13A-J are bar charts of proliferative responses of the TL3A6 clone to 200 decapeptide sublibraries with one defined amino acid position (20 for each of the 10 positions). The amino acids are indicated with one-letter codes on the X axis. Proliferation is shown as CPM induced by 200 μg/ml of the peptide libraries (average and SD of duplicate wells).

FIG. 13K is a block diagram illustrating important anchor sites for MBP binding to MHC II and a T cell receptor. The amino acids are indicated with one-letter codes.

FIG. 14 is a block diagram illustrating the process of training an artificial neural network (ANN) to classify 10-mer peptides according to their stimulatory potential for T cell.

FIG. 15 is a tree-based model of T cell stimulation. Sᵢ stands for the score for i^{th} position in 10-mer peptide.

### DETAILED DESCRIPTION OF THE PREFERRED EMBODIMENT

### Part 1

### Combinatorial Peptide Libraries and Biometric Score Matrices Permit the Quantitative Analysis of Specific and Degenerate Interactions Between Clonotypic TCR and MHC Peptide Ligands

The interaction of T-cell receptors (TCRs) with MHC-peptide ligands can be highly flexible, so that many different peptides are recognized by the same TCR in the context of a single restriction element. We provide a quantitative description of such interactions, which allows the identification of T-cell epitopes and molecular mimics. The response of T-cell clones (TCC) to positional scanning synthetic combinatorial libraries (PS-SCL) is analyzed with a mathematical approach that is based on a model of independent contribution of individual amino acids to peptide antigen recognition. This biometric analysis compares the information derived from these libraries composed of trillions of decapeptides with all the millions of decapeptides contained in a protein database to rank and predict the most stimulatory peptides for a given T-cell clone. We demonstrate the predictive power of the novel strategy and show that, together with gene expression profiling by cDNA microarrays, it leads to the identification of novel candidate autoantigens in the inflammatory autoimmune disease, multiple sclerosis.

### T-Cell Clones

T-cell clones (TCC) were established from peripheral blood or cerebrospinal fluid (CSF) lymphomononuclear cells by a split-well technique as previously described (Martin R. et al., 1992, J Immunol 148:1359). TCC GP5F11 was established from peripheral blood lymphomononuclear cells (PBMC) of a patient with multiple sclerosis (MS) using influenza virus hemagglutinin (HA) peptide 306-318 (PKYVKQNTLKLAT SEQ ID NO:3, single-letter amino acid code) as an antigen. The TCC is restricted by DRB1*0404. TCC TL3A6 was established with myelin basic protein (MBP) from PBMC of a patient with MS and recognizes the immunodominant epitope MBP₈₇₋₉₉(VHFFKNIVTPRTP) (SEQ ID NO:4) in the context of DR2a (DRα+DRB5*0101). The TCC has been extensively characterized for recognition of numerous altered peptides derived from MBP₈₇₋₉₉ as well as other molecular mimics (Hemmer B. et al., 1998, J Immunol 160:3631; Wilson D. B. et al., 1999, J Immunol 163:6424; Hemmer B. et al., 2000, J Immunol 164:861; Vergelli M. et al., 1997, J Immunol 158:3746; Vergelli M. et al., 1996, Eur J Immunol 26:2624). The TCR usage is TCRAV18 and TCRBV5S1. TCC CSF-3 was established with a lysate of B. burgdorferi from the CSF of a patient with chronic Lyme disease as described herein. The TCC recognizes several B. burgdorferi-derived as well as human peptides in the context of DR2b (DRα+DRB1*1501). The TCR usage is TCRAV13S2 and TCRBV14S1.

### Peptides and Peptide Combinatorial Libraries

Peptides were synthesized by the simultaneous multiple peptide synthesis method (Houghten R. A., 1985, PNAS USA 82:5131) and characterized using HPLC and mass spectrometry. A synthetic N-acetylated, C-amide L-amino acid combinatorial peptide library in a positional scanning format (PS-SCL; 200 mixtures in the OX₉ format, where O represents one of the 20 L-amino acids and X represents all of the natural L-amino acids except cysteine) was prepared as described (Pinilla C. et al., 1994, Biochem J 301:847).

### Proliferative Assays

The proliferation of TCC in response to PS-SCL or individual peptides was tested by seeding in duplicate 2×10⁴ T cells, 5×10⁴ irradiated PBMC with or without mixtures from PS-SCL or peptide. Proliferation was measured by [³H]-thymidine incorporation (Hemmer B. et al., 2000, J Immunol 164:861).

### Statistical Analysis and Model Building

A positional scoring matrix was generated by assigning a value of the stimulatory potential to each of the 20 defined amino acids in each position. The score Sᵢⱼ for each amino acid i at each position j was calculated as follows:

$${S}_{i j} = \frac{{L}_{i j} - B}{\sqrt{{( \text{std} ( {L}_{i j} ) )}^{2} + {( std ( B ) )}^{2}}}$$

where L equals the mean of replicate experimental measurements (c.p.m.), B stands for background noise,std(Lᵢⱼ) denotes the smoothed estimate of the standard deviation (SD) for each measurement using a locally weighted regression smoothing technique (S-plus package) based on the assumption that the SD is dependent on level of response. We call this the Z-index score due to its similarity to statistical Z ratios of means divided by their standard error (SE) values.

In an alternative score called stimulation index (S-index), we generated the score in each position by using the mean of duplicate c.p.m. values in the presence of mixtures from the PS-SCL fractions divided by the mean of duplicate values in the absence of mixtures from the PS-SCL. The S-index score appeared preferable when the PS-SCL spectrum of the c.p.m. value was more clearly defined.

A positional scoring matrix can be created by employing the smoothed data (see above), or other readouts of the data such as stimulation indices (test value divided by background value) or any other way to express the raw data without further manipulation (e.g., counts per minute from proliferative testing).

Under the assumption of independent contribution to stimulation, the predicted stimulatory potential of given peptide is the sum of the scores in each position. A 10-mer peptide sequence can be represented by a 20×10 matrix of Os and 1s (pᵢⱼ) where pᵢⱼ=1 if the i th amino acid (using the same order as for the rows of the scoring matrix) is in position j. Let Sᵢⱼ denote the components of the positional scoring matrix. Then the score for the peptide is:

$$S = {\sum }_{i = 1}^{20} {\sum }_{j = 1}^{10} {p}_{i j} {S}_{i j}$$

### Database Search

We wrote a Perl script to systematically search the GenPept database. A window with the same length of peptide as used in the PS-SCL was applied to slide over the available translated protein-coding sequences. The sum of the scores within the window was used as a ranking criterion. All peptides with scores higher than a threshold were output into a file. The threshold was chosen based on the statistical significance of the peptide score, compared to that for a random peptide. Those peptides were then sorted. Redundant peptides were removed. The database search can also be restricted to specific organisms (e.g. Homo sapiens or Influenza virus).

### Statistical Significance

We developed a statistical significance test of the hypothesis that the score for a peptide is no greater than would be expected if the peptide were obtained from 10 random draws of amino acids. Under the null hypothesis it is not assumed that all amino acids are equally likely, but rather the relative frequencies f₁, f₂, . . . , f₂₀ are derived from the database being searched. Under the null hypothesis, the distribution of S will be approximately normally distributed. The mean and the variance of this null distribution can be expressed as

$$\begin{matrix}m = {\sum }_{i = 1}^{20} {f}_{i} {\sum }_{j = 1}^{10} {S}_{i j} \\ var = E [ {S}^{2} ] - {m}^{2}\end{matrix}$$

The variance can be shown to equal:

$$\begin{matrix}var = {\sum }_{i = 1}^{20} {f}_{i} {\sum }_{j = 1}^{10} {S}_{i j}^{2} + 2 {\sum }_{j = 1}^{9} {\sum }_{{j}^{′} = j + 1}^{10} {m}_{j} {m}_{{j}^{′}} - {m}^{2} \\ where {m}_{j} = {\sum }_{j = 1}^{20} {f}_{ i} {S}_{i j} .\end{matrix}$$

The statistical significance of any score S can be approximated as

$$p = Φ ( \frac{m - S}{\sqrt{var}} ) ,$$

where Φ denotes the standard normal distribution function. This significance level does not, however, account for the number of 10-mer sequences contained in the database.

### Analysis of Gene Expression Using cDNA Microarrays

Brain tissue was obtained at autopsy from two MS patients. Patient W was a 46 year-old male with primary progressive MS (Becker K. G. et al., 1997, J Neuroimmunol 77:27), patient R was a 46 year-old female with relapsing-remitting MS. Normal white matter was dissected, post-mortem, from three non-diseased brains. RNA extracted from these three normal white matter samples was pooled, in equal amounts, for use in hybridization experiments. Lesions were identified by hematoxylin and eosin (H & E) and Luxol fast blue-periodic acid Schiff (LFB-PAS) staining of paraffin embedded sections. Further characterization of lesions was performed using immunohistochemistry for cell-specific antigens. All staging of lesions was performed as previously described (Lassmann H. et al., 1998, J Neuroimmunol 86:213). From the first patient, patient W, one acute (W1) and one chronically-active lesion (W2) were studied. From the second patient, R, sixteen chronic lesions were studied. These lesions had inflammatory cells present but the inflammatory cells were not participating in any form of on-going demyelination.

The detailed methodology of cDNA microarray analysis has been described in detail elsewhere (Whitney L. W. et al., 1999, Ann Neurol 46:425). Arrays for this study contained 2,889 human cDNAs that were primarily derived from I.M.A.G.E. consortium cDNA libraries (Lennon G. et al., 1996, Genomics 33:151). A list of genes present on the arrays can be found at http://intra.ninds.nih.gov/Biddison/cDNA_microarray.asp. [³³P]-dCTP-labeled cDNAs were produced by reverse transcriptase from RNAs obtained from individual MS lesions, pooled normal white matter, experimental allergic encephalomyelitis (EAE) and normal mouse brains, and hybridized to the cDNA microarrays. Hybridizations of RNA obtained from MS lesions and EAE brains were performed in two independent experiments, except for lesions R10, R11, and R16 in which enough RNA was obtained for only one hybridization. Quantitation of radioactivity bound to the arrays was performed on a Molecular Dynamics STORM phosphoimager (Molecular Dynamics, Sunnyvale, Calif.) at 50 μm resolution. All data was analyzed from the phosphoimager images using Pscan (Carlisle A. J. et al., 2000, Mol Carcinog 28:12, see also http://abs.cit.nih.gov/pscan). Pscan calculates spot intensities and compares spot intensities between samples, giving a ratio of gene expression between comparative samples. Using Pscan, spot intensities between arrays were automatically normalized to the median of all spot intensities on each individual array. Ratios of gene expression that were greater than two-fold were considered significant based on a 99% confidence interval (Chen Y., et al., 1997, Biomed Optics 2:364).

### Data Obtained with Combinatorial Peptide Libraries Suggest Different Levels of TCR Degeneracy for Different CD4⁺ T Cell Clones

Here, we sought to develop an approach that would combine the information generated from the screening of a decapeptide PS-SCL with all protein sequences in public databases. This strategy should allow the identification of the entire spectrum of stimulatory peptide ligands for a given TCC and the ranking of naturally occurring peptides with regard to predicted stimulation. The ultimate goal is to develop a methodology for identifying biologically relevant peptides for TCC of unknown specificity that have been isolated e.g. from a tissue.

Three CD4⁺ TCC were tested in proliferative assays with the 200 mixtures of the decapeptide PS-SCL. Two TCC had known specificity, one specific for influenza hemagglutinin (Flu-HA) (306-318) (T-cell clone GP5F11), and one for MBP₈₃₋₉₉ (T-cell clone TL 3A6). We also studied one clone of unknown specificity, which recognizes B. burgdorferi, the causative organism of Lyme disease (Tell clone CSF-3).

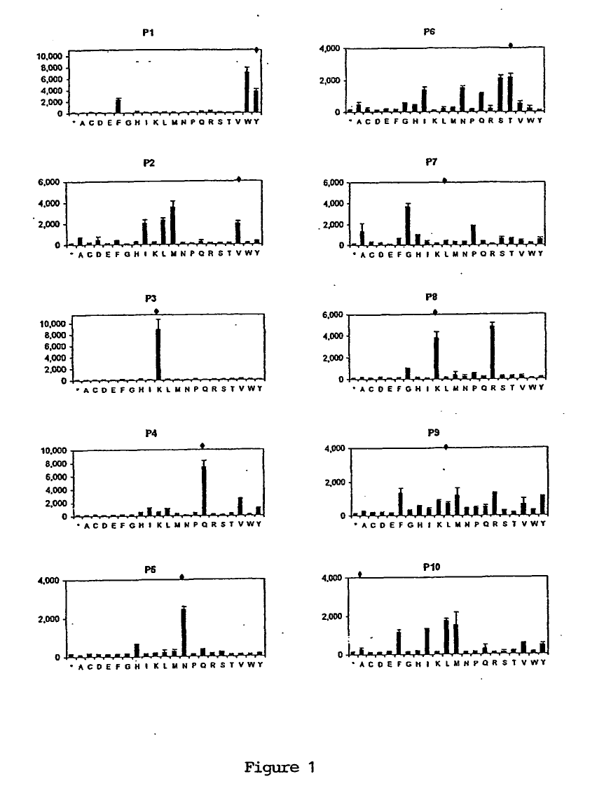

Data obtained with combinatorial peptide libraries suggest different levels of TCR degeneracy for different CD4⁺ T-cell clones. The stimulation profiles for TCC GP5F11 and TL 3A6 are shown in FIGS. 1A and B, respectively. The profile for CSF-3 is shown herein. The profile of TL3A6 shows that more than one mixture in several positions of the PS-SCL generated a clear proliferative response. The amino acids of MBP₈₉₋₉₈ are marked by diamonds (FFKNIVTPRT) (SEQ ID NO:2). Although the target amino acids correspond to the defined amino acid in the most stimulatory mixtures in most positions, this is not observed in certain positions, such as N in position 4 and P in position 8. In contrast, the profiles for GP5F11 and CSF-3 show a very different pattern with fewer but more differential activity between stimulatory and not stimulatory mixtures.

### Limitations of Motif Searches

Motif searches are widely used to search protein-databases in a non-quantitative manner. This approach was not successful, however, for identifying the known target peptides of the TL3A6 and GP5F11 clones. Motifs searches are generated from screening results of PS-SCL and contained in each position amino acids corresponding to mixtures with simulation index (S-index) greater than a specified threshold. Thresholds of 2 and 3 were used to generate the search motifs. The resulting motifs were then used to search the SwissPro and GenPept databases.

Tables 1 and 2 show the number of peptides which satisfied the motif searches and indicate whether the target peptide was identified. The target peptide was not found with either of the motifs for TL3A6 in either database. The target peptide for GPF11 was identified only when the search criterion was so permissive/lax that over 500 other peptides were also selected. Furthermore, the inability of motif searches to rank peptides renders it almost impossible to identify the most likely epitopes in a rational way and without synthesizing and testing very large numbers of individual peptides.

TABLE 1
Database search performed on SwissProt and GenPept to identify agonist peptides for TCC GP5F11

| | | SwissProt | | GenPept | | |
|---|---|---|---|---|---|---|
| | | Target | No. | Target | No. hits, viral | No. hits, |
| S-Index | Search Supermotif¹ | sequence | hits | sequence | DB | H. sapiens DB |
| >2 | [WYFRH]-[MLIVADFYH]-K-[QVILYHKPTM]-[NHQM]- | Yes | 513 | Yes | 560 | 177 |
| | [TSNIQGVAHM]-[GPAHFSTYVNQLICM]-[RKGPMTNVS]- | | | | | |
| | [FRMYKLVHQPNISWGA]-[LMIFVYQA] | | | | | |
| >3 | [WYFR]-[MLIVADF]-K-[QVILYHKP]-[NHQ]-[TSNIQGVAH]- | No | 82 | No | 23 | 34 |
| | [GPAHFSTYVNQL]-[RKGPM]-[FRMYKLVHQPNI]-[LMIFVY] | | | | | |

¹Amino acids corresponding to Flu HA(308-317)(YVKQNTLKLA) are shown in bold underlined characters. SwissProt contains 83,857 protein sequences (Mar. 3, 2000); GenPept viral database: 90,174 proteins (20,198,794 decamer peptides); Homo sapiens database: 43,795 proteins (13,879,822 decamer peptides).

TABLE 2
Database search performed on SwissProt and GenPept to identify agonist peptides for TCC TL3A6

| | | | | GenPept | | | |
|---|---|---|---|---|---|---|---|
| | | SwissProt | | | No. hits, | | |
| | | Target | | Target | H. sapiens | No. hits, | No. hits, |
| S-Index | Search Supermotif¹ | sequence | No. hits | sequence | DB | viral DB | bacterial DB |
| >2 | [WHYFARTLCGQVKN]-[KIFSRYLWMTAVN]- | No | 260,085 | No | 104,229 | 183,876 | 289,887 |
| | [KDLCGFVIYQNH]-[LKIMVSATDG]-[VMLIWYTR]- | | | | | | |
| | [VMILPTYSKWGEQNA]-[TSFVRWLQKGAPNY]- | | | | | | |
| | [KICTSPLFQMRAHW]-[FKRVPYLIH]- | | | | | | |
| | [TISVHWKMAFLR] | | | | | | |
| >3 | [WHYFARTLCG]-[KIFSRYL]-[KDLC]-[LKIMV]- | No | 797 | No | 285 | 502 | 776 |
| | [VMLIW]-[VMILPTYSK]-[TSFVRW]-[KICTSPL]- | | | | | | |
| | [FKRVPYL]-[TISVHW] | | | | | | |
| ¹Amino acids corresponding to MBP(89-98)(FFKNIVTPRT) are shown in bold underlined characters. SwissProt contains 83,857 protein sequences (Mar. 3, 2000); GenPept viral database: 90,174 proteins (20,198,794 decamer peptides); H. sapiens database: 43,795 proteins (13,879,822 decamer peptides); bacterial database: 111,807 proteins | | | | | | | |
| #(32,604,667 decamer peptides) | | | | | | | |

### Developing a Score Matrix-Based Approach for Predicting T-Cell Stimulatory Candidate Peptides

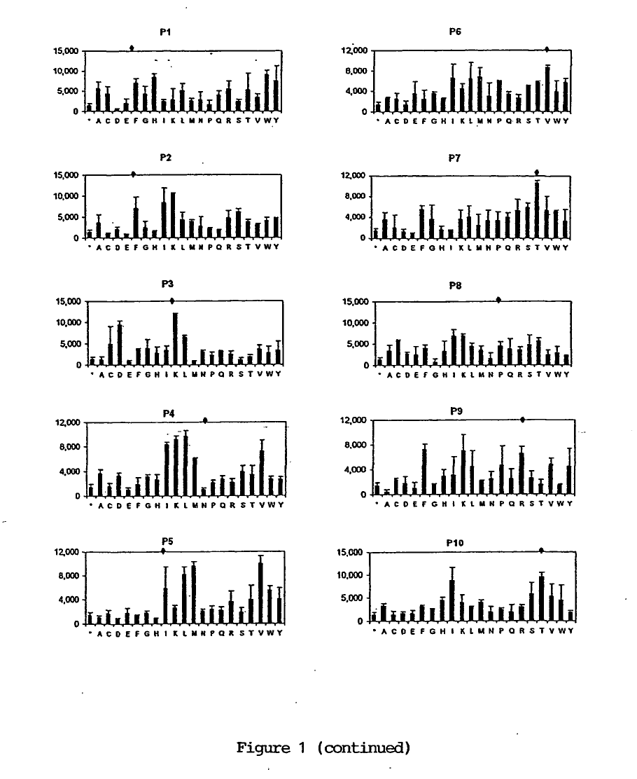

It is clear that a more systematic approach that employs all the data generated from the screening of PS-SCL needs to be developed for the search of databases. Our strategy is outlined in the flow diagram (FIG. 2).

We recently demonstrated that each amino acid within a peptide contributes to recognition almost independently and in an additive fashion, so that amino acid substitutions that abrogate recognition can be compensated for by highly stimulatory substitutions in other positions (Hemmer, B. et al., 1998, J Immunol 160: 3631). Thus, the overall stimulatory value of a peptide results from the combination of positive or negative effects of each of the amino acids. Based on these assumptions we could show that peptides that shared no amino acid in corresponding positions of their sequences could still be recognized by the same TCR (Hemmer, B. et al., 1998, J Immunol 160: 3631). Also, the findings that the specificity information derived from PS-SCL libraries is similar to that obtained with individual peptide analogs and the fact that highly active peptides can be identified allow the development of a new search algorithm.

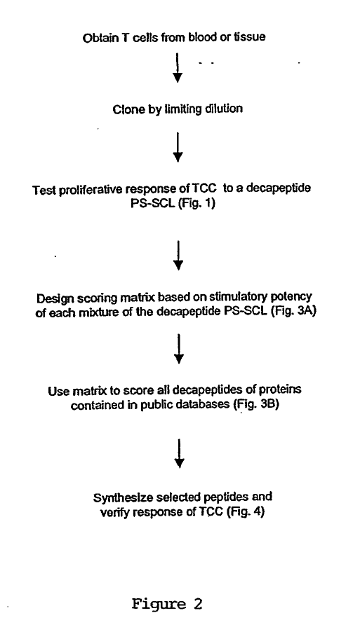

Our algorithm provides a predicted stimulatory score for the peptide of the same length as used in PS-SCL libraries. Based on the above assumptions, the peptide score is the sum of position specific scores of the component amino acids. The scoring is accomplished by calculation of a matrix in which the columns represent positions and the rows the 20 amino acids used in PS-SCL libraries. The scoring matrix entry for a particular amino acid in a specific position is based on the stimulation assay results for the mixture of PS-SCL corresponding to that amino acid defined in that position (FIG. 3A). The scoring matrix entry can either use the S-index or use the Z-index, which takes into account the experimental errors.

The matrix is then used to search for predicted stimulatory peptides in the public protein databases. By moving a decamer scoring window across the known protein sequences in one amino acid increments (FIG. 3B), a stimulatory score is calculated for all published 10-mer peptides, and then they are ranked accordingly. This strategy offers important advantages compared to motif searches: a) all the information derived from the PS-SCL screening is used, and the selection based on a cutoff of activity is not required; b) peptides are now ranked according to their predicted stimulatory score.

An example of a score matrix for one of the CD4⁺ TCC (GP5F11) is shown in FIG. 3A. The amino acids of the Flu-HA₃₀₈₋₃₁₇ peptide are boxed. Note that the amino acids of the target peptide sequence L in position P7 and A in P10 are below a S-index value of 3, thus explaining the failure of the motif search to find the target influenza peptide. The principle of the sliding decamer scoring window which is moved across a protein sequence in one amino acid increments is shown in FIG. 3B. Three decamer peptides within the Flu-HA₃₀₄₋₃₂₁ sequence are scored by adding the stimulatory values of the respective 10 amino acids. Note the drastic changes in stimulatory scores when the scoring window is moved one amino acid to the left (score 51.98) or to the right (13.7) as compared to the optimal register that is shown in the middle (score 256.01). These changes of the scores indicate that, as soon as both MHC and TCR contact positions which contribute most of the stimulatory activity are out of the correct register, the peptide may lose binding to the MHC and/or fail to stimulate the clone because the TCR contacts are not positioned properly.

### Testing the Score Matrix-Based Approach Using Clones with Known Specificity and with Synthesized Peptides

The effectiveness of this approach is demonstrated in Table 3. When the score matrices for clones TL3A6 and GP5F11 were used to score all peptides in the GenPept database, both the target peptides (MBP₈₉₋₉₈ peptide for TL3A6 and Flu-HA₃₀₉₋₃₁₈ for GP5F11) were correctly identified. The GenPept database (ftp://ftp.ncifcrf.gov/pub/genpept) was searched since it is substantially larger than SwissProt (http:/fw.vw.expasy.ch/sprot). The relative ranks obtained for the target peptides are given in Table 3.

TABLE 3
Database search performed on GenPept with a sum of S-index score
matrix

| TCC | Target Sequence | Rank in Database |
|---|---|---|
| GP5F11 | Yes | 6¹ |
| TL3A6 | Yes | 202² |

¹A total of 90,174 proteins scored in viral database (20,198,794 decamer peptides).
²A total of 43,795 proteins scored in H. sapiens database (13,879,822 decamer peptides).

For GP5F11, the rank among viral peptides is given; for TL3A6, we show the rank among human peptides. Consistent with previous observations with another autoreactive clone (Hemmer, B. et al., 1997, J Exp Med 185: 1651), MBP₈₉₋₉₈ was far from optimal, i.e. it ranked only 202^{nd} in the set of human peptides using the S-index matrix. In contrast, the target peptide Flu-HA₃₀₉₋₃₁₈ ranked as the 6^{th} highest scoring peptide for GP5F11 among viral proteins and 24^{th} when not only viral, but also human proteins were scored. This also suggests that molecular mimics that are potentially more stimulatory than the native foreign peptide can be identified.

We assessed the predictive power of the algorithm using synthesized peptides tested for stimulation of the three clones (76 peptides for GP5F11, 144 peptides for TL3A6, and 88 peptides for CSF-3). For the 2 TCC of known specificity, TL3A6 and GP5F11, the peptide was considered stimulatory if its EC₅₀ (concentration that yields at half-maximal stimulatory activity) was equal to or less than 10 times that of the target peptide (MBPg and Influenza virus HA₈₉₋₉₈ respectively). For CSF-3, the TCC of unknown specificity, the peptide was considered stimulatory if it activated the TCC with a Z-index>47.5 at any concentration between 0.001 and 100 μg/ml.

Table 4 shows the relationship between stimulatory potential predicted by the scoring matrices and actual measurement of TCC stimulation. Thresholds for matrix score prediction were based on ROC (Relative Operating Characteristic) analysis (Swets J. A., 1988, Science 240: 1285) to balance sensitivity and specificity. For clone CSF-3, for example, of the 62 peptides predicted to be stimulatory (have scores above the threshold of 47.5), 58 did stimulate the TCC (a positive predictive value of 58/62, or 93.5%). Of the 26 peptides predicted to be non-stimulatory, only 5 stimulated the TCC (negative predictive value: 21/26, 80.8%). The sensitivity for predictions with this clone was 92%; that is of the 63 peptides that actually stimulated the TCC, 58 were corrected predicted. The specificity was 84%; that is of the 25 peptides that did not stimulate the TCC, 21 were correctly predicted. While the sets of synthesized peptides are small compared to the number of peptides that would be predicted to be stimulatory, Table 4 documents the excellent sensitivity, specificity and negative predictive values for the three TCC.

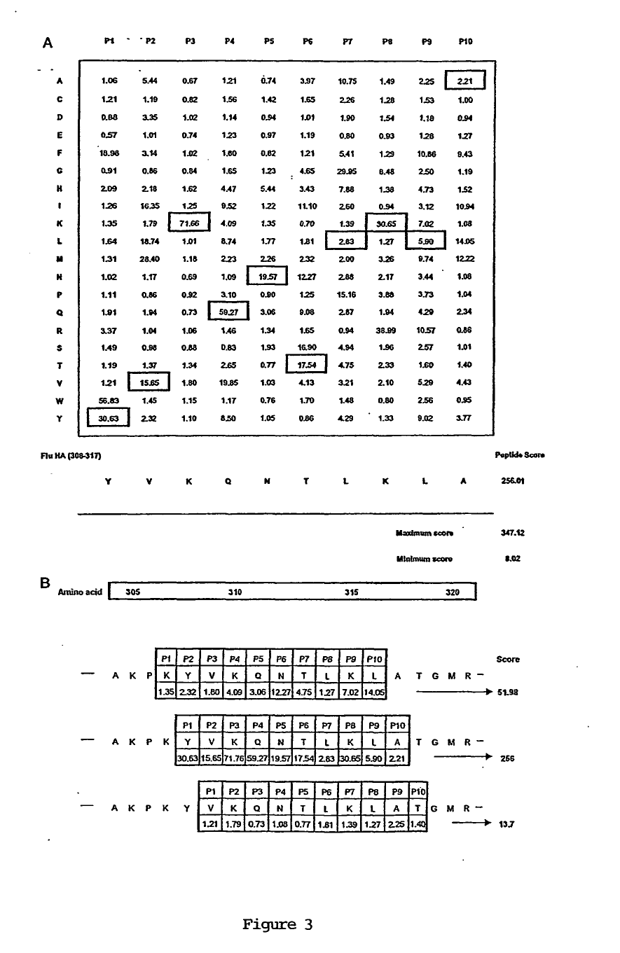

Table 5 shows the information on the 10 highest scoring peptides derived from B. burgdorferi database analysis for TCC CSF-3 with the half-maximal stimulatory value that was determined by dose-titration, proliferative experiments. Examples of the stimulatory activity of peptides predicted to activate TCC GP5F11 are shown in FIG. 4. Note that a predicted stimulatory peptide with optimal amino acids in each position (WMKQNIGRFL) (SEQ ID NO:9) and a higher score than the target peptide is in fact two orders of magnitude more potent than the target sequence. One of the shown peptides with a score of 132.40 ranks much lower than the putative stimulatory threshold for TCC GP5F11, and consequently it did not stimulate the clone. However, even a few high scoring peptides are not stimulatory from reasons that are currently under further investigation.

TABLE 4
Indices of the predictive power of the scoring matrix approach for the definition of the stimulatory potency of
antigenic peptides

| | TCC CSF-3 | | | | | | | | |
|---|---|---|---|---|---|---|---|---|---|
| | Matrix | | | TCC GP5F11 | | | TCC TL3A6 | | |
| | score | Matrix score | | Matrix | | | Matrix score | | |
| | >47.5 | <47.5 | Total | score > 220 | Matrix score < 220 | Total | >45.2 | Matrix score < 45.2 | Total |
| Experimental | | | | | | | | | |
| measurement | | | | | | | | | |
| Stimulatory | 58 | 585 | 63 | 38 | 4 | 42 | 20 | 8 | 28 |
| Nonstimulatory | 4 | 21 | 25 | 2 | 32 | 34 | 18 | 98 | 116 |
| Total | 62 | 26 | 88 | 40 | 36 | 76 | 38 | 106 | 144 |

| | | | |
|---|---|---|---|
| Sensitivity¹ | 58/63 (92) | 38/42 (90.5) | 20/28 (71.4) |
| Specificity² | 21/25 (84) | 32/34 (94.1) | 98/116 (84.5) |
| Positive | 58/62 (93.5) | 38/40 (95.0) | 20/38 (52.7) |
| predictive value³ | | | |
| Negative | 21/26 (80.8) | 32/36 (88.9) | 98/106 (92.5) |
| predictive value⁴ | | | |
| Overall accuracy⁵ | 79/88 (89.8) | 70/76 (92.1) | 118/144 (81.9) |

¹Fraction of all stimulatory peptides that is correctly identified.
²Fraction of all nonstimulatory peptides that is correctly identified.
³Probability that a peptide predicted to stimulate actually does so.
⁴Probability that a peptide predicted to be nonstimulatory actually does not activate the TCC.
⁵Fraction of all predictions that is correct.

TABLE 5
Information on the 10 highest scoring peptides derived from B. burgdorferi database analysis for TCC CSF-3

| | | Protein ID | | |
|---|---|---|---|---|
| Score | Sequence | No. | Protein Description | EC₅₀ μg/ml¹ |
| 54.82 | N N I Y K K A L I S | AE001155 | Hypothetical protein (section 41 of 70) of the complete genome | 1 |
| 54.14 | S N I I K S L S L F | AE001174 | Hypothetical protein (section 60 of 70) of the complete genome | 0.1-1 |
| 53.73 | S N I I K K T S E D | AE001169 | Similar to SP:P07017 (section 55 of 70) of the complete genome | 1 |
| 53.70 | F N I Y K R V V D N | AE001145 | Hypothetical protein (section 31 of 70) of the complete genome | 1 |
| 53.68 | N N I D K K V Y T N | AE001135 | (section 21 of 70) of the complete genome; similar to GB:Z32522 | 1-10 |
| 53.09 | F F I K K R S L I I | AE000785 | Hypothetical protein of plasmid Ip25 | 1 |
| 52.82 | R N I F K K T V E N | AE001130 | Similar to GB:L10328 (scetion 16 of 70) of the complete genome | >100 |
| 52.69 | S N I K S K L I L V | AE001146 | Similar to PID:1652132 (section 32 of 70) of the complete genome | 1 |
| 52.63 | Y N I I V S S L L L | AE001161 | Hypothetical protein (section 47 of 70) of the complete genome | 1-10 |
| 52.57 | D N I F K K E T L I | AE001165 | Similar to GB:L42023 (section 51 of 70) of the complete genome | 1 |

¹Peptide concentration inducing half-maximal proliferation.

### Combining Scoring Matrix Predictions of TCC Stimulation with cDNA Microarrays to Identify Biologically Relevant Candidate Peptide Mimics

The novel strategy described here allows us to find peptides from every known source that have stimulatory activity for the clone that was tested with PS-SCL. This leads to the problem of how one identifies from this wealth of data which peptides may be biologically relevant. In cases where the target antigen for the clone is not known or molecular mimics with potential relevance for an organ-specific disease are of interest, several strategies may be used.

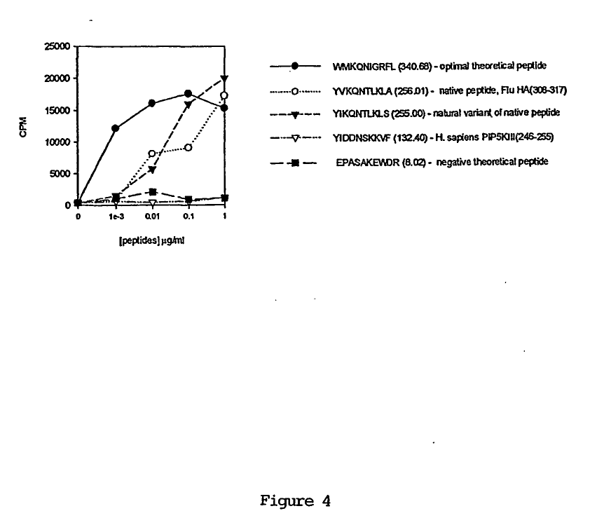

One approach to identification of proteins involved in autoimmune diseases is to examine expression of genes that are overexpressed in the target organ using cDNA microarray technology (Whitney et al. 1999, Ann Neurol 46: 425). We examined gene expression in 18 lesions from two MS patients and compared them to levels of gene expression in pooled normal white matter from three individuals with cDNA microarrays containing 2889 human genes. One of the genes that was overexpressed (>2-fold) in 17 of the 18 MS lesions examined was Titin (FIG. 5A), a giant muscle protein (Labeit and Kolmerer, 1995, Science 270: 293). When we asked which genes that are overexpressed in MS plaques are also identified as candidate epitopes/molecular mimics for CD4+ TCC that were tested with the PS-SCL (FIG. 5B), we identified peptides derived from the same interesting candidate, Titin, among the highest scoring peptides for both a CD4⁺ TCC recognizing the immunodominant myelin basic protein peptide (83-99) in the context of the MS-associated DR allele DRB5*0101, but also for the B. burgdorferi-specific TCC CSF-3 (FIG. 5C). Titin, a giant muscle protein (Labeit and Kolmerer, 1995, Science 270: 293), is surprisingly overexpressed in MS brain tissue, and the identification of titin-derived peptides as candidate molecular mimics for two TCC that are potentially pathogenic in two different CNS inflammatory/autoimmune disorders, i.e. MS and chronic CNS Lyme disease, offers unique opportunities to study the involvement of such candidate antigens in the pathogenesis of these diseases.

### T-Cell Receptors and MRC-Peptide Ligands

The experiments presented here have been conducted in order to better understand, measure and predict both specific and degenerate interactions between clonotypic T-cell receptors and MHC-peptide ligands. For this purpose, an approach was devised that would allow us to a) to describe in a quantitative way the complex interactions of the trimolecular antigen recognition complex, and b) to identify the spectrum of stimulatory ligands for individual T cell clones with high predictive accuracy. We employed combinatorial peptide libraries and biometric strategies in conjunction with large scale database searches to achieve this goal and could show for the first time that T cell recognition can be predicted in quantitative terms. This study builds on and expands previous investigations on the flexibility and degeneracy of TCR recognition of antigen. A role for degenerate T cell recognition has been postulated for such diverse immunological phenomena as thymic selection (Bevan M. J. 1997, Immunity 7:175), peripheral T-cell survival (Hemmer B. et al., 1998, Immunol Today 19:163), protection from infectious diseases, and induction of autoimmunity (Hemmer B. et al., 1998, Immunol Today 19:163; Gran B. et al., 1999, Ann Neurol 45:559). It was previously shown that peptide combinatorial libraries in the positional scanning format can be used to define the spectrum of agonist ligands for clonotypic TCR (Hemmer B. et al., 1998, Immunol Today 19:163; Pinilla C. et al., 1999, Curr Opin Immunol 11:193). In recent studies, we showed that functional responses elicited in CD4⁺ TCC by PS-SCL could be utilized to build motifs for database searches and thus identify a spectrum of ligands of different potency for clonotypic TCR (Hemmer B. et al., 1997, J Exp Med 185:1651; Swets J. A. 1988, Science 240:1285). In the present study, we confirmed that functional T cell responses can be elicited by PS-SCL from certain CD4⁺ TCC specific for both foreign (FIG. 1A) and self (FIG. 1B) antigens. We then utilized a matrix-based methodology for the analysis of the experimental data generated with the PS-SCL (FIG. 2). This methodology is based on a model of independent and additive contribution of each amino acid in the peptide sequence to the interactions with both the TCR and the MHC molecule (Hemmer B. et al., 1998, J Immunol 160:3631). While numeric matrices (Hammer J. et al., 1994, J Exp Med 180:2353) and other mathematical approaches based on independent amino acid contribution to antigenicity have been previously utilized to describe the interaction of antigenic peptides with specific MHC molecules (Brusic V. et al., 1998, Bioinformiatics 14:121; Gulukota K. et al., 1997, J Mol Biol 267:1258), the present study fills the important gap of applying a quantitative, matrix-based model to the interaction of an MHC-peptide ligand (keeping the MHC molecule constant) with a specific, clonotypic TCR using the data generated from PS-SCL. The biometrical analysis described here systematically compares the information derived from a PS-SCL composed of trillions of decapeptides with all the decapeptides (13,879,822 for a H. sapiens database and 20,198,794 for a viral database) contained in a public protein database to rank and predict the most stimulatory peptides for a given TCC. The predictions based on this methodology are so accurate (Tables 3 and 4, FIG. 4) that they actually lend strong support to an additive, combinatorial model of peptide antigenicity. Available TCR crystal structures indeed suggest that peptides may modulate the preexisting affinity between MHC and TCR that is based on a large contact surface between these two components of the trimolecular complex (Garboczi D. N. et al., 1996, Nature 384:134; Garcia K. C. et al., 1996, Science 274:209). It should be noted that this model does not contradict, but indeed extends and develops the concept of primary and secondary TCR contacts (Degano M. et al. 2000, Immunity 12:251; Kersh G. J., P. M. Allen. 1996, J Exp Med 184:1259). In fact, although complex substitutions of amino acids along the entire sequence of the peptide can lead to molecular mimicry in the absence of any sequence homology (Hemmer B. et al., 1998, J Immunol 160:3631), the relative weight of different amino acids in each position of the peptides sequence is apparent from the experimental data (FIGS. 1A and B).

An important application of the above described model is that one can identify peptide ligands for a specific TCR by searching public database not only with MHC- and TCR anchor motifs (Wucherpfennig K. W., J. L. Strominger. 1995, Cell 80:695) or motifs obtained from PS-SCL data (Hemmer B. et al., 1997, J Exp Med 185:1651; Hemmer B. et al., 1998, Immunol Today 19:163), but also using the scoring matrix derived from the screening of a PS-SCL composed of trillions of peptides (FIGS. 3A and B). We also illustrate the limitations of using motifs derived from PS-SCL screening to identify TCR agonist peptides. Such a strategy does not fully utilize the information generated by screening specific TCR with PS-SCL. Therefore, the native ligand may not be found if the motif is not sufficiently degenerate (Table 1, S-index>3; Table 2, S-index>3; S-index>2), or if even one of the positions does not contain the amino acid that appears in the native sequence. Another advantage in the identification of T-cell epitopes is that one can rank the predicted stimulatory peptides according to their score. This is of great practical value when the number of candidate peptides is very high (Table 2) and one needs criteria to select which of the identified candidate peptides should be synthesized and actually tested with the TCC. In addition to identifying promptly the target peptide sequences (Table 3), one can then synthesize and test a feasible number (hundreds) of candidate peptides to confirm their stimulatory activity (examples in FIG. 4; see also Table 4). Interestingly, we confirmed our previous observation that for autoreactive TCC, the ligand used to establish and expand the TCC is often a suboptimal one, consistent with the notion that high-affinity self-reactive TCC are deleted in thymic selection (Nossal G. J. 1994, Cell 76:229). Whereas for autoreactive TCC we often found natural ligands derived from foreign or even self antigens whose potency was several orders of magnitude higher than that of the native peptide (Hemmer B. et al., 1997, J Exp Med 185:1651), for TCC GP5F11 and other TCC specific for foreign antigens the native ligand was much closer to the optimal one (Table 3) (Bielekova B., et al., 1999, J Neuroimmunol 100:115; Hemmer B. et al., 1998, J Immunol 160:5807). Although more potent synthetic ligands could be designed based on the deconvolution of the PS-SCL data (Pinilla C. et al., 1999, Curr Opin Immunol 11:193; Hemmer, B. et al., 2000, J Immunol 164:861) (e.g., peptide WMKQNIGRFL, SEQ ID NO:9 in FIG. 4), naturally occurring superagonists were rare. The fact that foreign antigen-specific TCC may recognize their antigenic peptides as highly potent ones is consistent with an efficient immune response required to eliminate infectious agents.

This study adds a new and important contribution to the definition and prediction of T-cell epitopes using synthetic combinatorial libraries (Pinilla C. et al., 1999, Curr Opin Immunol 11:193; Hiemstra H. S. et al., 2000, Curr Opin Immunol 12:80). It should be noted that many of the previous approaches to the identification of T-cell epitopes were based on the prediction of which peptides would be good binders for specific MHC/HLA molecules (Hammer J. et al., 1994, J Exp Med 180:2353; Southwood S. et al., 1998, J Immunol 160:3363). Since only a fraction of the potential MHC-binding peptides is a T-cell epitope for an individual TCR, these approaches provide information that is specific for particular MHC molecules, but cannot predict which fraction of the peptides that bind a restriction element are actually stimulatory for a TCR with its unique structural features. Conversely, TCR ligands are not always high affinity MHC binders (Muraro P. A. et al., 1997, J Clin Invest 100:339). The approach presented in this study takes into account the whole trimolecular complex of T-cell activation by reading out a functional T-cell response. This requires a certain degree of MHC-peptide binding as well as the interaction of the MHC-peptide ligand with a specific TCR. When both are considered, the overall accuracy of T-cell epitope predictions is far superior to previously adopted methods (Table 4), although further improvements are currently being pursued. This is particularly helpful when the protein(s) recognized by a TCC is/are not known. Indeed, less than a third of the peptides that were identified and found to be stimulatory by the PS-SCL and scoring matrix approach would have been predicted to be good MHC binders based on a recently published MHC-binding prediction algorithm (Sturniolo T. et al., 1999, Nat. Biotechnol. 17:555).

Finally, we show that combining the above described methodology with the use of cDNA microarrays to assess differential gene expression in pathological and normal tissue of two patients with MS led to an interesting candidate molecule (Titin, thus far only known as an important component of skeletal muscle) (Labeit S., B. Kolmerer. 1995, Science 270:293) that is overexpressed in MS plaques and is recognized by a B. burgdorferi-specific TCC (FIG. 5). Preliminary pathological studies by immunohistochemistry indicate the expression of an isoform of this molecule in the pathologic, as opposed to normal white matter tissue, but further work to define its role is clearly needed. Thus, the combination of two powerful methodologies can guide the discovery of candidate autoantigens that would otherwise not easily be identified by either approach.

In summary, we describe a methodology, PS-SCL-based biometrical analysis for ligand identification, that is consistent with a combinatorial model of TCR activation by antigenic peptides and allows the identification of T-cell epitopes for both autoreactive and foreign antigen-specific TCC with unprecedented efficacy. We have used the same approach successfully for the prediction and identification of antigens by CD8⁺ TCC. For the first time, recognition of antigens by clones of unknown specificity can be decrypted. This is an important advance in the study of autoimmune disease, where one tries to suppress specific immune responses, as well as for infectious and neoplastic diseases, where a stimulation of specific responses by vaccines is pursued. Furthermore, it is important to note that this approach can be used to identify ligands within proteins in public databases for any molecular interaction that has been or can be studied with PS-SCLs composed of not only L-amino acids, but also other strings of similar building blocks, e.g., sugar molecules, lipid molecules, nucleotides, D-amino acids and other synthetic derivatives and compounds that can be employed following similar principles, i.e., additive and independent contribution of each building block to the interaction.

### Part 2

### Identification of Candidate T-cell Epitopes and Molecular Mimics in Chronic Lyme Disease

Elucidating the cellular immune response to infectious agents is a prerequisite for understanding disease pathogenesis and designing effective vaccines. In the identification of microbial T-cell epitopes, the availability of purified or recombinant bacterial proteins has been a chief limiting factor. In chronic infectious diseases such as Lyme disease, immune-mediated damage may add to the effects of direct infection by means of molecular mimicry to tissue autoantigens. Here, we describe a new method to effectively identify both microbial epitopes and candidate autoantigens. The approach combines data acquisition by positional scanning peptide combinatorial libraries and biometric data analysis by generation of scoring matrices. In a patient with chronic neuroborreliosis, we show that this strategy leads to the identification of potentially relevant T-cell targets derived from both Borrelia burgdorferi and the host. We also found that the antigen specificity of a single T-cell clone can be degenerate and yet the clone can preferentially recognize different peptides derived from the same organism, thus demonstrating that flexibility in T-cell recognition does not preclude specificity. This approach has applications in the identification of ligands in infectious diseases, tumors and autoimmune diseases.

### Introduction

T-lymphocyte responses are central in acquired, antigen-specific immune responses. After antigen recognition, CD4⁺ T-cells are activated to exert effector functions such as cytokine production and cytotoxicity, and to provide help for both the cellular and humoral arms of the immune response. Short peptides derived from the processing of antigenic proteins are presented to T-cells by antigen-presenting cells in the context of major histocompatibility molecules (MHC; HLA in humans) (Germain, R. N., 1994 Cell 76: 287-299). The identification of immunodominant peptides is an essential step in understanding the pathogenesis of such diverse processes as the response to infectious agents, immune surveillance to cancer, and autoimmune diseases. It is also a prerequisite for the rational design of vaccines and specific immunomodulatory therapies. In each of the three disease categories mentioned above, the complexity of the structures to be recognized (infectious agents, tumor antigens and normal self antigens) makes identification of immunodominant antigens difficult. The use of recombinant proteins and sets of overlapping peptides that cover their entire sequences has been a useful but limited tool in defining T-cell epitopes (Walden, P. 1996 Curr Opin Immunol 8: 68-74). In fact, even a small virus presents several proteins as immunological targets, and each of them can have multiple overlapping epitopes that may be recognized differently in individuals with different immunogenetic backgrounds. The complexity of antigen recognition increases with proteins derived from larger viruses, bacteria, parasites and humans.

Here we present a new approach to identify antigenic epitopes in complex organisms. We used chronic Lyme disease of the central nervous system (CNS) as a model of infection by an organism, Borrelia burgdorferi, which elicits a complex T-cell-mediated immune response in a well-defined organ compartment (Haass A., 1998 Curr Opin Neurol 11: 253-258). The complexity of B. burgdorferi, its antigenic variability and the limited availability of its proteins in forms suitable for T-cell studies have all hindered the analyses of immunity to this organism (Sigal L. H., 1997 Annu Rev Immunol 15: 63-692). In addition to the direct pathogenic effect of B. burgdorferi, cross-recognition of self antigens by B. burgdorferi-specific T-cell clones has been postulated to be involved in the development of chronic lesions in the CNS as well as in other affected organs (Sigal L. H., 1997 Semin Neurol 17: 63-68; Gross D. M. et al., 1998 Science 281: 703-706). Here, we use a strategy that includes positional scanning synthetic combinatorial peptide libraries (Pinilla C. et al., 1992 Biotechniques 13: 901-905; Hemmer, B. et al., 1998 J Pept Res 52: 338-345) (PS-SCL) and biometric data analysis (PS-SCL-based biometric data analysis for ligand identification) to identify a spectrum of ligands for single, in vivo-expanded T-cell clone isolated from the cerebrospinal fluid (CSF). We identify peptide ligands for a T-cell clone that was most likely expanded in vivo by exposure to B. burgdorferi and grown in vitro with a lysate of the same organism. Many of these peptides are derived from different proteins of B. burgdorferi. In addition, we identify candidate autoantigens that are potent agonists for this T-cell clone and are recognized in part also by peripheral blood lymphocytes, indicating that they may serve as targets of immunopathologic injury and contribute to inflammatory tissue damage during chronic CNS Lyme disease.

### Case Report

A 33-year-old white male, an avid recreational hunter from Wisconsin, presented in 1993 with meningoencephalitis and cerebral vasculitis. Both Lyme ELISA and IgG western blot analysis were positive by Centers for Disease Control criteria (J Am Med Assoc, 1995, 274: 937), and there was specific intrathecal antibody production (CSF/serum index of 2.05) against B. burgdorferi. His vasculitis and meningoencephalitis resolved upon antibiotic therapy (ceftriaxone 2 g intravenously per day for 4 weeks). In the ensuing 4 years, three similar episodes were treated with ceftriaxone. During the fourth episode, he was found to have mediastinal adenopathy; a biopsy of this showed noncaseating granulomas. He has persistent high titers of B. burgdorferi-specific antibodies in serum and CSF and a positive intrathecal antibody index. His HLA type is: A1, 26; B7, 57; CW6, 7; DR2 (DRB1*1501), DQw6 (DQB1*0602).

### Borrelia burgdorferi Antigens

For the production of B. burgdorferi lysate, collected, low-passage B. burgdorferi JD1 were washed three times in 0.01 M phosphate-buffered saline (PBS), pH 7.2, sonicated, and reconstituted in PBS. Recombinant purified unlipidated outer surface protein A and B were a gift from J. Dunn (Brookhaven National Laboratory).

### Combinatorial Peptide Libraries and Decapeptides

A synthetic N-acetylated, C-amide L-amino acid combinatorial peptide library in a positional scanning format (200 mixtures in the OX9 format, where O represents one of the 20 L-amino acids and X represents all of the natural L-amino acids except cysteine) was prepared as described (Pinilla C. et al., 1994, Biochem J 301: 847-853). Each OX₉ mixture consists of 3.2×10¹¹ (19⁹) different decamer peptides in approximate equimolar concentration (0.26 M). Individual peptides were synthesized by the simultaneous multiple peptide synthesis method (Houghten, R. A. 1985, PNAS USA 82: 5131-5135). The purity and identity of each peptide were characterized using an electrospray mass spectrometer interfaced with a liquid chromatography system

### T-Cell Lines and Clones

T-cell lines and clones were established by a split-well technique (Martin, R. et al. 1992, J Immunol 148: 1359-1366) from CSF lymphomononuclear cells. Cells were seeded in 96-well plates at a concentration of 1×102 to 1×10³ cells per well with 2×10⁵ autologous, irradiated (3,000 rad) peripheral blood mononuclear cells and a 1:200 dilution (volume/volume) of B. burgdorferi lysate. These cells were cultured in IMDM containing 100 U/ml penicillin/streptomycin, 50 μg/ml gentamycin, 2 mM L-glutamine (all from BioWhittaker, Gaithersburg, Md.) and 5% human serum. Cells were expanded by weekly re-stimulation with B. burgdorferi lysate (1:200 dilution, volume/volume), 20 U/ml hrIL-2 (National Cancer Institute, National Institutes of Health) and autologous or HLA-DR-matched irradiated peripheral blood mononuclear cells.

### Flow Cytometry

The clonality of T-cell lines and clones was analyzed by 22 monoclonal antibodies specific for human TCRBV chain families (Immunotech, Marseille, France). T-cells were divided into 11 aliquots and stained with 5 μl of a mixture of antibodies containing a PE-Cy-conjugated monoclonal antibody against CD3 and a combination of two monoclonal antibodies against TCRBV labeled with FITC and PE, respectively. After samples were incubated 30 min on ice and washed twice in PBS+1% FCS, the fluorescence intensity and number of positive cells were determined on a FACScalibur (Becton Dickinson, Franklin Lakes, N.J.).

### Proliferative Assays

The proliferation of T-cell clones in response to PS-SCL or individual peptides was tested by seeding in duplicate 2×10⁴ T-cells, 5×10⁴ irradiated peripheral blood mononuclear cells with or without 200 μg/ml PS-SCL or peptide (Hemmer, B. et al. 1998, J Pept Res 52: 338-345). Proliferation was measured by ³H-thymidine incorporation (Hemmer, B. et al. 1997, J Exp Med 185: 1651-1659). Experiments were repeated at least twice. HLA restriction was determined by using bare lymphocyte syndrome cells transfected with DR2a (DRB5*0101) or DR2b (DRB1*1501) (provided by G. Nepom, University of Washington, Seattle) (Kovats, S. et al. 1994, J Exp Med 179: 2017-2022). The proliferation of peripheral blood mononuclear cells was measured in an IL-7-modified 7-day primary proliferation assay. Peripheral blood mononuclear cells (1×10⁵ per well; 10 wells each and 10 control wells per plate) were seeded with 10 ng/ml IL-7 and 15 μg/ml peptide. Then, 1 μCi ³H-thymidine/well was added at day 7 for 12 h before radioactivity was measured. Wells were considered positive if the counts per minute exceeded the average background proliferation+3 s.d. (Reece J. C. et al., 1993, Immunol 151: 6175-6184).

### T-Cell-Receptor Signaling

T-cells (1×10⁶) were added to peptide-pulsed bare lymphocyte syndrome cells (2×10⁶) and, after centrifugation (10 s at 300 g), were incubated at 37° C., washed once with PBS and incubated for 25 min on ice in lysis buffer (1% Nonidet-P40, 10 mM Tris-HCl, pH 7.2, 140 mM NaCl, 2 mM EDTA, 5 mM iodoacetamide, 1 mM Na₃VO₄ and complete protease inhibitor ‘cocktail’; Boehringer). After nuclear debris were removed, proteins in lysate supernatants were immunoprecipitated by incubation at 4° C. for 12 h with rabbit antibody against ZAP-70 (provided by L. Samelson, National Institute of Child Health and Human Development, National Institutes of Health). Proteins were separated by SDS-PAGE and immunoblotted with 4G10, a mouse monoclonal antibody against phosphotyrosine (Upstate Biotechnology, Lake Placid, N.Y.).

### RT-PCR, Single-Strand Conformation Polymorphism and Sequencing

The single-strand conformational polymorphism clonotype analysis was done as described (Yamamoto, K. et al., 1996, Hum Immunol 48:23). mRNA was isolated from 1×10⁴ CSF mononuclear cells or 1×10⁶ CSF-3 T-cells by the Quick Prep Micro mRNA purification kit (Pharmacia). One-third of the mRNA was converted into cDNA (First Strand DNA Synthesis Kit; Pharmacia), which was amplified by PCR in a 50-1 volume using 30 pmol of primer sets consisting of a TCRBC-specific primer and of each TCRBV-specific primer (Illes, Z. et al., 1999, J Immunol 162: 1811-1187). Each PCR cycle comprised 30 s at 94° C., 30 s at 60° C. and 1 min at 72° C., with 40 cycles for CSF mononuclear cells and 32 cycles for CSF-3. Amplicons were diluted 1:2 in denaturing solution (95% formamide, 10 mM EDTA, 0.1% bromophenol blue and 0.1% xylene cyanole) and heated at 90° C. for 2 min. PCR products (2 μl) were separated by electrophoresis (35 W for approximately 2 h) in a 4% polyacrylamide gel containing 10% glycerol. DNA was then transferred onto Immobilon-S (Millipore, Bedford, Mass.). The TCR amplicons were visualized by the NEBlot Phototype Detection Kit (NEB), using a biotinylated internal TCRBC probe. Using TOPO™ cloning (Invitrogen, Carlsbad, Calif.), TCRBV14-positive PCR products of CSF3 were inserted into the pCR 2.1-TOPO plasmid vector. Plasmid DNA was isolated from colonies of transformed bacteria by Wizard Plus Minipreps DNA Purification System (Promega), and was sequenced at the National Institute of Neurological Disorders and Stroke DNA Sequencing Facility.

### Cytokine Production

T-cells (2×10⁵) and autologous irradiated (3,000 rad) cells (1×10⁶) were cultured in the presence or absence of B. burgdorferi lysate (1:200 dilution, volume/volume). Supernatants were collected after 48 h, and the levels of IFN-γ, TNF-α, GM-CSF, IL-4, IL-5 and IL-10 were determined by ELISA (Biosource, Camarillo, Calif.).

### Scoring Matrix and Database Searches

A scoring matrix was generated by assigning numerical values to the stimulatory potency of defined amino acids at each position in the mixtures (based on one representative PS-SCL experiment of five). The numerical score in each position was calculated as the difference between the proliferation (in counts per minute) in the presence of peptide mixtures and the proliferation in the absence of peptides divided by a smoothed estimate of the standard error of background and peptide-induced proliferation. The stimulatory potential of a peptide can be predicted by summing the scores associated with each amino acid in each position of the peptide. For a PS-SCL experiment using peptides 10 amino acids in length, a search of the GenPept protein database was conducted by moving a 10-amino-acid peptide ‘window’ along all sequences and scoring all 10-amino-acid peptides. All high-scoring peptides 10 amino acids in length were thereby identified.

### Borrelia burgdorferi-Specific T-Cell Clones

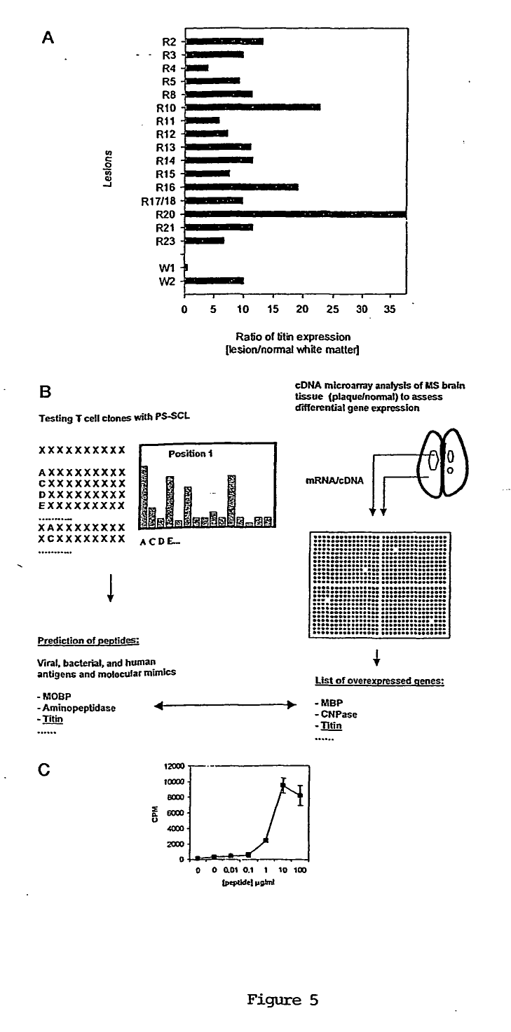

We isolated lymphocytes from the CSF of a patient with chronic neuroborreliosis, seeded them by limiting dilution and stimulated them with whole B. burgdorferi lysate. According to Poisson statistics, the estimated precursor frequency of B. burgdorferi-specific T-cell clones in the CSF was approximately 1 in 800 (Moretta A. et al., 1983, J Exp Med 157:743-754). We expanded growing colonies and characterized them with respect to surface markers, Tell-receptor-variable β (TCRBV) chain expression, antigen specificity, and cytokine release (Table 6). Each of these T Hell cultures was CD4⁺, responded specifically to B. burgdorferi lysate and, with one exception (CSF-1), secreted substantial amounts of gamma interferon (IFN-γ), tumor necrosis factor (INF)-α, granulocyte-monocyte colony-stimulating factor (GM-CSF), and interleukin (IL)-10, but no IL-4 or IL-5 (T-helper cell type 1-like phenotype). This is consistent with previous results obtained from organ-infiltrating T-cell clones isolated from patients with Lyme disease (Yssel H. et al. 1991 J Exp Med 174: 593-601). We analyzed the single-strand conformational polymorphism patterns of unmanipulated CSF cells to identify the most important in vivo-expanded T-cell clonotypes (Yamamoto K. et al., 1996, Hum Immunol 48: 23-31; Illes Z. et al., 1999, J Immunol 162: 1811-1187). T-cells expressing a limited number of T-cell receptor Vβ family chains (TCRBV) were clonally expanded in the CSF (FIG. 6). Approximately 5% of unstimulated CSF CD4⁺ T-cells expressed TCRBV14. The single-strand conformational polymorphism pattern of one isolated T-cell clone (CSF-3) corresponded to one of the clonotypes, indicating in vivo expansion. We also determined the TCRBV amino-acid junctional sequence of the T-cell clone (FIG. 6). After preliminary screening of all T-cell cultures for their response to the peptide libraries, we concentrated our study on T-cell clone CSF-3 because it was clonally expanded in vivo (FIG. 6), it produced high amounts of T-helper-cell-1 cytokines (Table 6) and it gave the most potent and reproducible responses to the peptide libraries.

TABLE 6
T-cell receptor variable β (TCRBV) chain usage and cytokine
production of T-cell lines and clones isolated from CSF

| | TCRBV | | | GM- | | | |
|---|---|---|---|---|---|---|---|
| TCC/TCL | usage | IFN-γ | TNF-α | CSF | IL-4 | IL-5 | IL-10 |
| CSF-1 | 21 | 91 | 375 | 228 | 201 | <dl | 297 |
| CSF-2 | nd | 640 | 581 | 370 | <dl | <dl | 54 |
| CSF-3 | 14 | 1104 | 211 | 218 | <dl | <dl | 121 |
| CSF-4 | 20 | 263 | 85 | 271 | <dl | <dl | 209 |
| CSF-5 | 1, 2, 3, | 90 | 130 | 199 | <dl | <dl | 165 |
| | 5S3, 22 | | | | | | |
| CSF-6 | 5S1 | nd | nd | nd | nd | nd | nd |
| CSF-7 | 20 | 487 | 504 | 444 | <dl | <dl | 60 |
| CSF-10 | 2, 22 | 75 | 718 | 52 | <dl | <dl | <dl |

Data represent pg/ml. TCC, T-cell clone; TCL, T-cell line (containing more than one T-cell receptor V-P clonotype); <dl, below detection limit; nd, not done.

### Identification of B. burgdorferi Epitopes and Mimics

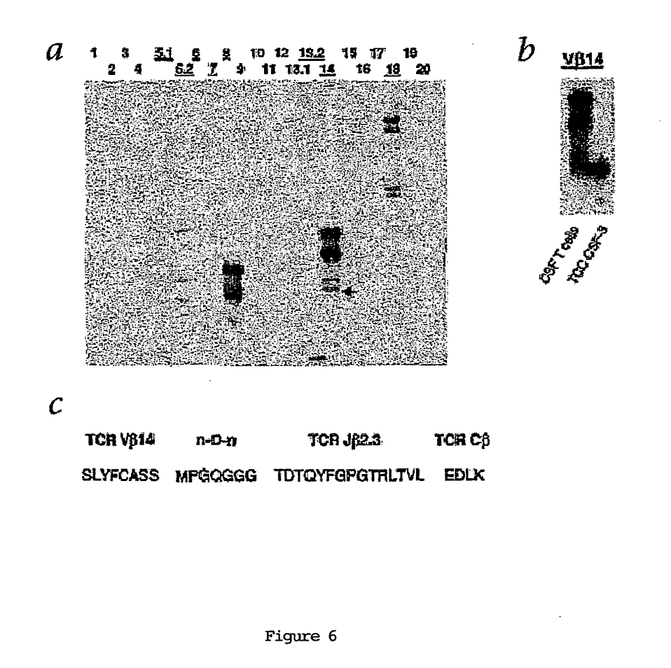

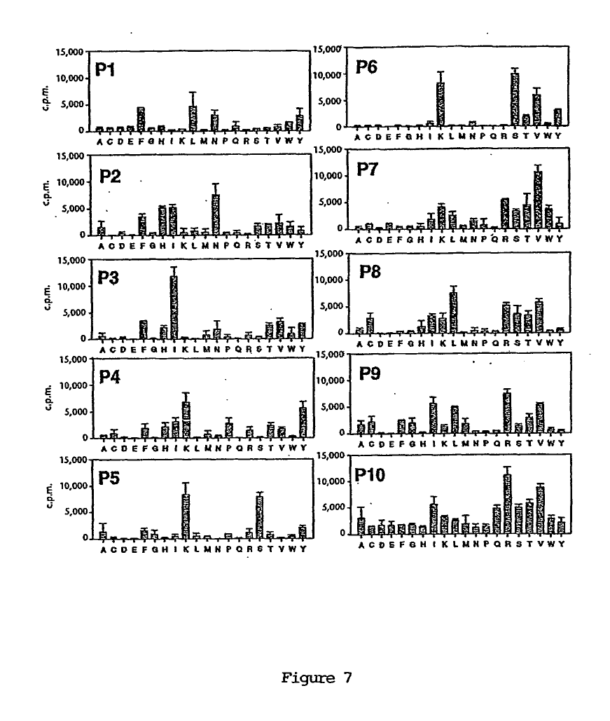

We used a new method that combines the use of decapeptide PS-SCL and biometric data analysis to identify B. burgdorferi epitopes recognized by clones that are likely to be expanded in vivo, and mimic peptides derived from self antigens. We tested the T-cell clone with a decapeptide PS-SCL composed of 200 peptide mixtures as described (Hemmer, B. et al., 1998 J Pept Res 52: 338-345; Hemmer, B. et al., 1998 Immunol Today 19: 163-168). This method allows the determination of favored and optimal amino acids for each position of putative TCR epitopes. At each position, only a few mixtures were strongly stimulatory (FIG. 7). We used the results obtained in the peptide library experiments to examine the public databases with a new strategy. Based on the assumption of independent and additive contribution of each position of a peptide (Hemmer B. et al., 1998 J Pept Res 52: 338-345; Hemmer B. et al., 1998 Immunol Today 19: 163-168), we generated a scoring matrix by transforming the stimulatory potency of each of the 20 L-amino acids in each of the 10 positions of the decamer libraries into numerical values. We then calculated the score for an individual peptide by adding the individual stimulatory values of the 10 amino acids in a decamer. We designed a program to use the matrix to score all the overlapping 10-amino-acid peptides in the GenPept database and thus identify sequences with the highest stimulatory values. We searched the entire B. burgdorferi genome (Fraser C. M. et al., 1997 Nature 390: 580-586) as well as databases compiling all known human and viral proteins (GenPept release 107.0), and ranked peptides 10 amino acids in length according to the highest numerical score (Table 7). The percentage of peptide sequences 10 amino acids in length with a high score was substantially greater in the complete B. burgdorferi genome than in the human and viral databases (Table 7), indicating a higher probability for the T-cell clone to recognize B. burgdorferi antigens. Accordingly, the score distribution for all the peptides contained in the B. burgdorferi genome, in contrast to the complete genomes of one closely related organism, Treponema pallidum, and two more distant ones, Mycobacterium tuberculosis and Escherichia coli was skewed to higher values (Table 7 and FIG. 8). The approach presented here to analyze these highly complex data represents a refinement of a strategy previously used to identify agonist peptides for CD4⁺ T-cell clones (Hemmer B. et al., 1998 J Pept Res 52: 338-345; Hemmer B. et al., 1997 J Exp Med 185: 1651-1659). In fact, the use of a ‘supermotif’ derived from the PS-SCL experiments to scan the public databases led to the identification of 25 peptides, only one of which was stimulatory to the T-cell clones. This peptide (Table 10, 52′) had the highest score value among the 25 identified. The new method proved successful in identifying B. burgdorferi-derived as well as mimic peptides with agonist properties for T-cell clone CSF-3.

TABLE 7
Database search for 10-amino-acid peptides predicted to be stimulatory for the TCC CSF-3

| | | | Calculated | Predicted | | |
|---|---|---|---|---|---|---|
| | Proteins | 10-amino-acid | stimulatory | stimulatory (% | | Actually |
| | analyzed | peptides scored | value (score) | of total scored) | Synthesized | stimulatory^{a} |
| Borrelia | 2,217 | 541,107 | 40-45 | 2,702 (0.499) | 1 | 1 |
| Burgdorferi | | | 45-50 | 387 (0.071) | 2 | 2 |
| Peptides | | | >50 | 30 (0.005) | 29 | 29 |
| | | | Total | 3,119 (0.576) | 32 | 32 |
| Viral peptides | 65,429 | 15,085,689 | 40-45 | 13,146 (0.087) | — | — |
| | | | 45-50 | 1,718 (0.011) | 1 | 1 |
| | | | >50 | 125 (0.001) | 12 | 12 |
| | | | Total | 14,989 (0.099) | 13 | 13 |
| Human | 33,344 | 10,487,433 | 40-45 | 8,253 (0.089) | — | — |
| Peptides | | | 45-50 | 985 (0.009) | — | — |
| | | | >50 | 68 (0.001) | 11 | 11 |
| | | | Total | 9,306 (0.089) | 11 | 11 |

Representative stimulatory peptides, Tables 8-10.
^{a}All peptides stimulatory at ≦μg/ml except one.

TABLE 8
Sequence, potency and function of Borrelia burgdorferi peptides

| | Potency | | | | | |
|---|---|---|---|---|---|---|
| | | % of | | | | |
| | EC₅₀ | Max. | PB | | | Reference or |
| Sequence | μg/ml^{a} | response^{b} | PP^{c} | Definition | Notes | submission |
| (26) QAIGKKTQNN | <0.001 | 80.8 | 3 | nd | Function unknown | TIGR (Fraser, |
| | | | | | | 1997) |
| (28) TLITKKISAI | <0.001 | 65.9 | 1 | Outer surface protein C (OspC) | Outer membrane lipoprotein (24 kDa; | (Padula, 1993) |
| | | | | | early antibody response to Bb) | |
| (30) LNIKNSKLEI | <0.001 | 64.2 | 0 | p22 lipoprotein | Serologically recognized in Lyme | (Lam, 1994) |
| | | | | | disease | |
| (37) FNIIKVHSSL | 0.001-0.01 | 69.3 | 1 | Sensory transduction histidine kinase | Related to chemotaxis operon in Bb | TIGR (Trueba, |
| | | | | (putative) | | 1997) |
| (38) YNIKKIKVED | 0.1-1 | 62.7 | 1 | DNA-directed RNA polymerase | 129.8 kDa, putative involvement in | (Alekshun, |
| | | | | (rpoB) | antibiotic resistance | 1997) |
| (42) LNITSSSYLF | 1-10 | 71.4 | 0 | Oligopeptide ABC transporter | Oligopeptide permease | TIGR (Bono, |
| | | | (OppAIV) | | | 1998) |
| (46) ENIKKILLRE | 0.1-1 | 65.1 | 3 | Chromosomal replication initiator | Product of dnaA gene | (Old, 1993) |
| | | | | protein | | |
| (47) NNIKSKVDNA | 1-10 | 80.6 | 0 | P37 immunogenic protein (putative) | Function unknown | TIGR |
| (54) FFIKKRSLII | 1-10 | 59.8 | 5 | nd | Function unknown | TIGR |
| (57) SNIIKKTSED | 0.1-1 | 65.2 | 4 | Methyl accepting chemotaxis protein | Function unknown in Bb | TIGR |
| | | | | (mcp5) | | (Sprenger, |
| | | | | | | 1997) |
| (59) NNIYKKALIS | 0.1-1 | 68.1 | 3 | Unique Bb integral membrane protein | Function unknown in Bb | TIGR |

TABLE 9
Sequence, potency and function of human autoantigenic mimics

| | Potency | | | | | |
|---|---|---|---|---|---|---|
| | EC₅₀ | max. | PB | | | Reference or |
| Sequence | μg/ml^{a} | response^{b} | PP^{c} | Definition | Notes | submission |
| (23) YSICKSGCFY | 0.1-1 | nt | nt | Myelin-associated oligodendrocyte | Third most abundant protein in CNS | (Yamamoto, 1994) |
| | | | | basic protein (MOBP) | compact myelin | |
| (61) LHIISKRVEA | 0.1-1 | 70.0 | 0 | Titin | Giant protein involved in muscle | (Labeit, 1995) |
| | | | | | ultrastructure and elasticity | |
| (62) SFIYSVVCLV | 0.1-1 | 75.7 | 9 | Somatostatin receptor isoform 1 | Somatostatinergic neurotransmission | (Yamada, 1992) |
| | | | | | modulates cognitive function and may | |
| | | | | | be defective in Alzheimer's disease | |
| (63) GHIKKKRVEA | 1-10 | 56.5 | 0 | Transforming growth factor-beta3 | Potent immunosuppressive cytokine; | (Derynck, 1988) |
| | | | | (TGF-β3) | TGF-β3 is mainly expressed in cells of | |
| | | | | | mesenchymal origin | |
| (64) FNITSSTCEL | 0.1-1 | 66.3 | 1 | Human C-C chemokine receptor | Lymphoid-specific EBV-induced G | (Schweickart, 1994; |
| | | | | type 7 precursor | protein-coupled receptor; upregulated | Sallusto, 1998) |
| | | | | | during dendritic cell maturation | |
| (66) ENVKKSRRLI | 0.1-1 | 64.1 | 0 | Interleukin-1 (IL-1) receptor | Receptor for IL-1α and IL-1β; type I | (Sims, 1989) |
| | | | | type 1, precursor | membrane protein; binding to agonist | |
| | | | | | leads to activation of NF-κB | |
| (71) DNITSSVLFN | 0.1-1 | 60.6 | 5 | Aminopeptidase A | Cleaves acidic amino acids off N- | (Nanus, 1993; |
| | | | | | terminus of polypeptides (angiotensin | Li, 1993) |
| | | | | | II, IL-8, CCK-8); may cleave both IL-7 | |
| | | | | | and IL-7R (N-terminal E); EC 3.4.11.7; | |
| | | | | | genomic structure similar to CD10, | |
| | | | | | CD26; marker of immature B cells, | |
| | | | | | upregulated by IL-7, viral | |
| | | | | | transformation, type I interferons. | |

TABLE 10
Sequence, potency and function of viral mimic peptides

| | Potency | | | | | |
|---|---|---|---|---|---|---|
| | | % of | | | | |
| | EC₅₀ | max. | PB | | | Reference or |
| Sequence | μg/ml^{a} | response^{b} | PP^{c} | Definition | Notes | submission |
| (52′) FNIIKSLLGG | 1-10 | nt | nt | Human Herpesvirus 6 (HHV-6) | homology to human adeno-associated | (Thompson, 1991) |
| | | | | protein U94 | virus type 2 (AAV-2) rep 68/78 gene | |
| | | | | | product; important for the life cycle of | |
| | | | | | HHV-6 and for host CD4 cell | |
| (74) PNITFSVVYN | 0.1-1 | 78.3 | 0 | Human adenovirus 40 and 41 | Ligand between adenovinis capsid and | (Pieniazek, 1990) |
| | | | | (fiber protein 2) | host cell receptor | |
| (75) FNITSSIRNK | 1 | 63.5 | 1 | HIV-1 envelope glycoprotein | HIV variant | (Gao, 1996) |
| (77) ENIYYSSVRT | 1-10 | 100 | 3 | HHV-7, strain JI, ribonucleotide | Catalyzes the first reaction in the DNA | J Nicholas, Dept. |
| | | | | reductase, large subunit | replication pathway (EC 1.17.4.1) | Oncology, JHU |
| Tables 8-10: | | | | | | |
| Numbers in parentheses, peptide identification numbers. | | | | | | |
| ^{a}Concentration inducing half-maximal proliferation for each peptide. | | | | | | |
| ^{b}Percentage of highest value for proliferative response in one representative experiment (100% = 28,255 counts per minute (c.p.m.) for peptide 77; proliferation in the absence of peptide = 3,081 c.p.m.). | | | | | | |
| ^{c}Primary proliferative response of peripheral blood mononuclear cells to the peptides. Data represent the number of wells showing a positive proliferative response, as indicated by c.p.m. higher than the average + 3 s.d. of 10 wells cultured in the absence of peptides (Table 8 and 10: proliferation in the absence of peptides | | | | | | |
| #2,899 ± 1,417 c.p.m.; Table 9: proliferation in the absence of peptides 1,306 ± 582 c.p.m.). | | | | | | |

nd - not defined.
nt - not tested in the same experiment.
TIGR The Institute for Genomic Research, Rockville, MD.

### Clonal and Bulk T-Cell Responses to the Peptides

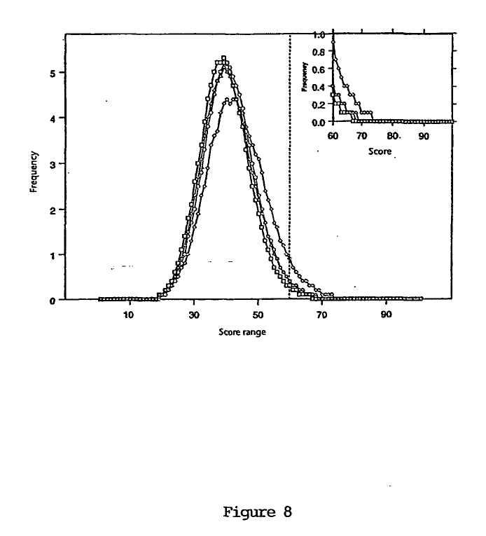

We synthesized 32 B. burgdorferi peptides, 11 human and 13 viral mimic peptides that were predicted to be highly stimulatory for T-cell clone CSF-3; all 56 elicited specific proliferation (Table 7 and FIG. 9a). The stimulatory potency of some of the B. burgdorferi peptides was highest, as indicated by the low EC₅₀ values (concentration inducing half-maximal proliferation) (Table 8, peptides 26-37). We compiled the sequence and protein source as well as the biological roles (if known) of some of the identified peptides (Tables 8-10). Low-scoring peptides derived from myelin basic protein and other proteins were non-stimulatory. To estimate how many other peptides could be stimulatory to the T-cell clone, we investigated artificial neural network and decision tree analyses to improve the predictive power of the model as described herein.

To determine whether the identified peptides are stimulatory not only for T-cell clone CSF-3, but also for bulk peripheral blood lymphocytes in this patient, we seeded 10 wells (1×10⁵ cells/well) with each of the peptides and equal numbers of control wells/plate in primary proliferative assays and analyzed how many showed stimulation three standard deviations above background (Tables 8-10). Many of these peptides induced proliferation in bulk peripheral blood lymphocytes similar to standard recall antigens, indicating a high precursor frequency of T-cells specific to these peptides. The primary amino-acid sequence differs substantially among many of the peptides, confirming previous observations that little or no sequence homology may be required for cross-recognition (Hemmer B. et al., 1998 J Immunol 160: 3631-3636). There was HLA DRB1*1501-restricted recognition of the peptides and of the B. burgdorferi lysate by clone CSF-3 (FIG. 9b). To assess the agonist potency of the identified B. burgdorferi and mimic peptides (that is, their potential for full activation of the T-cell clone), we tested their effect on early TCR signaling events. There was a fall agonist response, as indicated by the appearance of higher-molecular-weight TCR4-chain phospho-isoforms (p38) and the recruitment of active ZAP-70 (Hemmer B. et al., 1998 J Immunol 160: 5807-5814) (FIG. 9c), for two representative peptides (59, derived from a membrane protein of B. burgdorferi, and 71, derived from human aminopeptidase A).

To exclude the possibility of nonspecific stimulation of different clones by the peptides identified for T-cell clone CSF-3, we tested their ability to stimulate three other B. burgdorferi-specific T-cell clones established from the same patient (CSF-1, CSF-4 and CSF-7) and one MBP-specific T-cell clone derived from a DR2-positive individual. There was no response to the peptides.

### PS-SCL Based Biometric Analysis for Ligand Identification

Here we have shown that a strategy combining the use of PS-SCL and biometrical analysis for ligand identification was effective in identifying specific target epitopes for an organ-infiltrating, in vivo-expanded T-cell population that had been stimulated with a crude lysate from a complex infectious organism. Evidence from both pathological (Oksi J. et al., 1996, Brain 119: 2143-2154) and immunological studies (Sigal L. H., 1997, Semin Neurol 17: 63-68; Garcia-Monco J. C. & Benach J. L., 1997, Semin Neurol 17: 57-62) has shown that although the presence of the infectious agent itself is pathogenic in the earlier stage of Lyme disease, autoimmune mechanisms may be involved in progression of the tissue damage when the bacterium is no longer detectable in affected organs. In chronic, treatment-resistant Lyme arthritis (Steere A. C. et al., 1990, N Engl J Med 323, 219-223; Lengl-Janssen B., et al., 1994, J Exp Med 180, 2069-2078; Kamradt T. et al., 1996, Infect Immun 64, 1284-1289), a T-cell response to the immunodominant epitope of B. burgdorferi outer surface protein A may induce chronic inflammation through molecular mimicry to the integrin LFA-1 (Gross D. M. et al., 1998, Science 281: 703-706). In chronic CNS lesions, vasculitis and lymphocytic infiltrates both indicate involvement of cell-mediated autoimmunity, but little is known about the specific B. burgdorferi antigens that may be involved in CNS Lyme disease. Moreover, information is scarce on which CNS antigens may be relevant as target autoantigens in this condition (Sigal L. H., 1997, Semin Neurol 17: 63-68; Martin R. et al., 1988, Ann Neurol 24: 509-516). By adopting the strategy described here, we sought to identify both types of antigens. We used a lysate of B. burgdorferi that should contain a large number of immunodominant as well as minor antigenic determinants of the spirochete. The combinatorial library method did not require assumptions about the nature of the proteins and peptides to be identified (Hemmer B. et al., 1998, J Pept Res 52: 338-345). To use these data with as few ‘pre-assumptions’ as possible for database searches, we applied a new biometrical method and transformed the experimental results into a score matrix that represented in numerical values the stimulatory potency for each amino acid and each position of a putative peptide. This method allowed us to define peptide epitopes for a TCR of unknown specificity. In so doing, we identified many B. burgdorferi epitopes. Some are derived from well-characterized proteins that are known to be a target in the immune response to the organism (OspC, p22, p35, p37), whereas the function of others is either putative or unknown (Table 8). It will be useful to determine whether antigen-presenting cells can process and present these peptides from the native proteins (OspC, p22, p35 and p37). At present, these experiments are hindered by the fact that only a few B. burgdorferi proteins have been isolated in native or recombinant form and that they show some degree of sequence polymorphism. However, the MHC-restricted activation of T-cell clone CSF-3 by B. burgdorferi lysate-pulsed processing-deficient cells (HLA-DR2-transfected bare lymphocyte syndrome cells, Kovats, S. et al. 1994, J Exp Med 179: 2017-2022, FIG. 9b), indicates that antigen processing may not be as essential as anticipated. Indeed, MHC-restricted activation of the T-cell clone by the lysate indicates that stimulatory peptides may be contained in the lysate or that some B. burgdorferi proteins may bind to and be recognized in the context of DRB1*1501 in native conformation and unprocessed (Vergelli, M. et al., 1997, Eur J Immunol 27: 941-951). In addition to peptides derived from several B. burgdorferi proteins, we also identified many mimic sequences, some of which are interesting candidate targets for an autoimmune response in the CNS (Table 9 and 10). Experiments are envisioned as establishing whether the above peptides elicit an inflammatory CNS disease in experimental models.

A salient immunological finding here was the identification of several different B. burgdorferi epitopes that are stimulatory for a single in vivo-expanded T-cell clone. This is likely to reflect a high degeneracy of antigen recognition by T-cell clone CSF-3. The recent speculation that a single T-cell clone may recognize as many as 10⁵ to 10⁶ peptides, as supported by several lines of evidence, including our data, has important biological implications (Mason, D., 1998, Immunol Today 19: 395-404). Without flexibility/degeneracy in T-cell recognition, the limited number of T-cells in an organism (in the range of 10⁸ in a mouse) would allow recognition of only a very small fraction of the peptides contained in a mixture of randomized peptides 10 amino acids in length (20¹⁰, or 1.024×10¹³, different sequences). The immune system would thus be flawed, with substantial ‘holes’ in the T-cell repertoire, if recognition were not degenerate. The in vivo expansion of T-cell clones such as CSF-3 (FIG. 6) may be facilitated by their capacity to recognize several different peptides derived from a particular organism (Table 7 and FIG. 8). The biological advantage of such a response may lie in an increased likelihood of early T-cell activation during infection, whereas a highly stringent interaction with one or a few peptides would be expected to be less efficient. These data indicate that the flexibility in antigen recognition is not incompatible, but instead can coexist, with a preferential, ‘extended specificity’ for antigens derived from a particular organism. Three other T-cell clones obtained from the CSF of the patient did not respond to any of the peptides identified for CSF-3. Thus, the set of different peptides recognized is very specific for that clone. It is likely that degenerate antigen recognition may also indicate a propensity of certain T-cell clones to respond to a higher number of mimic peptides derived from self or viral proteins expressed in the CNS (Tables 9 and 10) and therefore, in the appropriate context, impose a higher risk for autoimmune responses (Gran B., et al., 1999, Ann Neurol 45: 559-567).

Although more extensive studies are needed to document the biological importance of the identified peptides for other patients with CNS Lyme disease, the following evidence indicates disease relevance in our patient: expansion of T-cell clone CSF-3 in the affected organ compartment (the CSF; FIG. 6); production of T-helper-cell-1-type cytokines (Gross D. M. et al., 1998, J Immunol 160: 1022-1028) (Table 6) and restriction by DRB1*1501, a class II allele that has been associated with CNS inflammation in multiple sclerosis (Allen M. et al. 1994, Hum Immunol 39 41-48) (FIG. 9b); the high potency of many of the peptides in activating the clone (FIG. 9a and Tables 8, 9, 10) and their full agonist quality (Hemmer, B. et al., 1998. J Immunol 160: 5807-5814) (FIG. 9c); and the presence of high numbers of peripheral blood T-cell precursors specific for many of the identified peptides.

In conclusion, we have presented a method, PS-SCL-based biometrical analysis for ligand identification, that allows ‘decryption’ of the epitope specificities for both the infectious organism and candidate autoantigens in a complex disease. This strategy has many applications that could not be implemented before. This knowledge is envisioned as being translated into the design of vaccines that can stimulate the immune response to infectious agents and tumors or suppress the response to autoantigens in autoimmune diseases.

### Part 3

### Biometrical Analysis of Screening Data of Hexapeptide PS-SCL in the mu Opioid Radioreceptor Assay

The screening and deconvolution of positional scanning combinatorial libraries (PS-SCLs) in opioid receptor binding assays have led to the identification of new opioid peptides having high activity (Dooley, C. T. and R. A. Houghten, 2000, Biopolymers 51:379-390). The objective of this analysis was to determine if the use of the biometrical analysis would lead to the prediction of the known natural ligands, which are present in protein databases. Table 11 shows the inhibitory activity of the mixtures of the hexapeptide PS-SCL. They have been sorted based on their activity within each position and the amino acid corresponding to one of the known ligands, M-enkephalin (in shading). It is clear that in most of the positions those amino acids defined one of the most active mixtures. This screening profile as well as a number of new non acetylated hexapeptides identified through the deconvolution of this PS-SCL have been reported (Dooley, C. T. and R. A. Houghten, 1993, Life Sci 52:1509-1517).

TABLE 11
Binding affinities (IC₅₀) for mixtures of hexapeptide PS-SCL

| Position | | Position | | Position | | Position | | Position | | Position | |
|---|---|---|---|---|---|---|---|---|---|---|---|
| 1 | μM | 2 | μM | 3 | μM | 4 | μM | 5 | μM | 6 | μM |
| Y | 63 | G | 51 | F | 30 | F | 41 | Y | 94 | F | 113 |
| F | 155 | V | 172 | G | 54 | Y | 146 | F | 102 | V | 114 |
| R | 313 | R | 210 | M | 136 | M | 172 | M | 135 | R | 159 |
| G | 490 | P | 216 | Y | 141 | G | 173 | R | 170 | K | 191 |
| L | 526 | F | 236 | L | 152 | H | 183 | L | 179 | K | 206 |
| P | 568 | M | 256 | I | 240 | R | 197 | I | 265 | T | 240 |
| I | 585 | A | 380 | R | 254 | A | 203 | H | 286 | H | 268 |
| H | 697 | L | 400 | A | 281 | L | 218 | K | 296 | L | 291 |
| M | 727 | H | 574 | N | 317 | P | 220 | T | 330 | I | 313 |
| A | 770 | T | 598 | H | 369 | K | 273 | V | 355 | G | 314 |
| V | 835 | S | 609 | P | 405 | V | 284 | N | 403 | A | 350 |
| K | 1123 | K | 613 | S | 446 | I | 314 | A | 405 | S | 360 |
| N | 1218 | I | 634 | Q | 460 | S | 322 | S | 422 | V | 400 |
| S | 1297 | V | 688 | K | 466 | N | 347 | P | 426 | N | 405 |
| T | 1297 | N | 692 | V | 481 | T | 388 | G | 480 | P | 408 |
| E | 1307 | Q | 855 | T | 529 | Q | 470 | Q | 536 | Q | 495 |
| D | 1349 | D | 1158 | E | 1292 | E | 1089 | D | 1127 | D | 1351 |
| Q | 1534 | E | 1408 | D | 1432 | D | 1209 | E | 1300 | E | 1425 |

To carry out the biometrical analysis a Z-scoring matrix (see below) was derived from the screening values (Table 12). This Z-scoring matrix was used to score and rank all the hexapeptides within the human database, which is composed of 49,765 proteins that in turn represent about 13 million hexapeptides.

TABLE 12
Z-scoring matrix derived from screening values of DCR 132

| | Position | | | | | |
|---|---|---|---|---|---|---|
| | 1 | Position 2 | Position 3 | Position 4 | Position 5 | Position 6 |
| A | 2.46 | 0.97 | 0.86 | 0.57 | 1.16 | 1 |
| D | 3.68 | 3.45 | 3.94 | 3.55 | 3.09 | 3.82 |
| E | 3.8 | 3.66 | 3.44 | 3.14 | 3.09 | 4.24 |
| F | 0.47 | 0.61 | 0.08 | 0.11 | 0.28 | 0.31 |
| G | 1.31 | 0.13 | 0.14 | 0.53 | 1.33 | 0.87 |
| H | 1.9 | 1.63 | 1.07 | 0.52 | 0.79 | 0.72 |
| I | 1.61 | 1.46 | 0.7 | 0.89 | 0.76 | 0.87 |
| K | 3.09 | 1.29 | 1.24 | 0.64 | 0.8 | 0.54 |
| L | 1.41 | 0.84 | 0.41 | 0.75 | 0.49 | 0.77 |
| M | 1.75 | 0.57 | 0.39 | 0.31 | 0.39 | 0.59 |
| N | 3.04 | 1.48 | 0.82 | 0.67 | 1.14 | 1.07 |
| P | 1.6 | 0.56 | 1.15 | 0.47 | 1.12 | 1.15 |
| Q | 4.15 | 2.22 | 1.37 | 1.08 | 1.48 | 1.44 |
| R | 0.82 | 0.49 | 0.75 | 0.41 | 0.45 | 0.36 |
| S | 3.39 | 1.45 | 1.24 | 0.65 | 1.18 | 1.09 |
| T | 3.39 | 1.6 | 1.7 | 1.36 | 1.01 | 0.72 |
| V | 1.71 | 1.83 | 1.23 | 0.88 | 0.93 | 1.27 |
| Y | 0.16 | 0.49 | 0.4 | 0.42 | 0.27 | 0.32 |

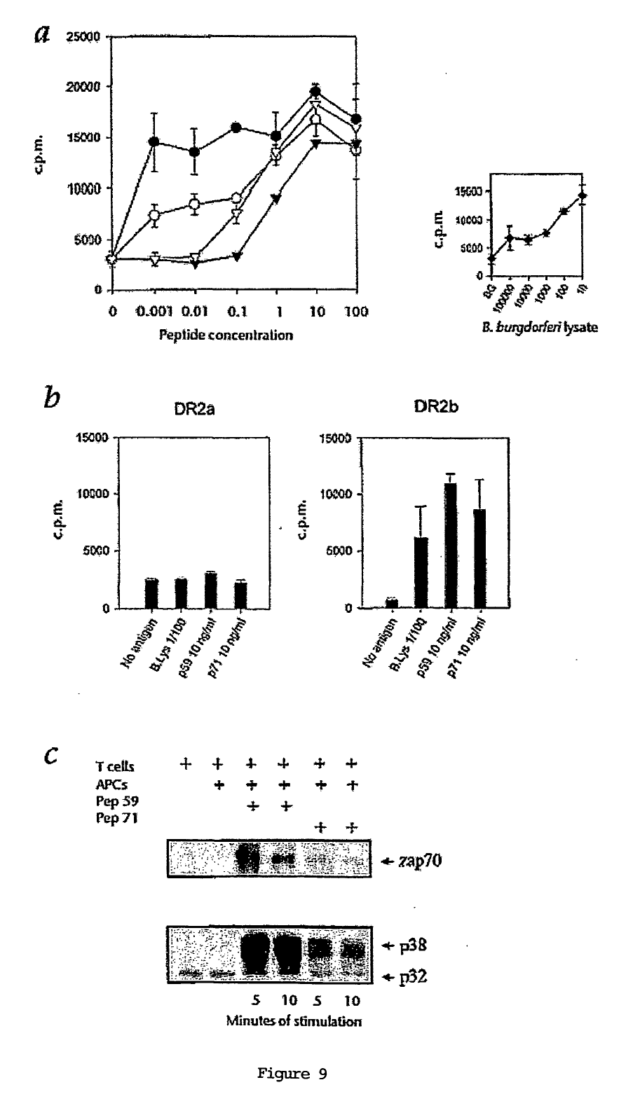

The results of this analysis include the scoring distribution and a list of sequences for the peptides with the highest scores. FIG. 10 represents the scoring distribution of all the hexapeptides in the human database. It can be seen that the majority of the peptides have scores much lower than the highest scores, and the number of peptides with the highest scores is low as compared to the total number of peptides scored. Also, table 13 below shows the rank, score and protein source of five known opioid ligands. It is clear that the known ligands ranked within the top 50 peptides out of 13 million hexapeptides that were scored, demonstrating the predictive value of the biometrical analysis for G protein coupled receptors.

TABLE 13
Known opioid ligands ranked within the top 50 peptides out of
13 million hexapeptides in human database

| Rank | Score | Peptide Sequence | | | | | | Protein Source |
|---|---|---|---|---|---|---|---|---|
| 3 | −1.29 | Y | G | G | F | M | R | V00510/J00123 |
| | | | | | | | | Preproenkephalin |
| 7 | −1.39 | Y | G | G | F | L | R | K02268 Enkephalin B |
| 9 | −1.47 | Y | G | G | F | M | K | V00510/J00123 |
| | | | | | | | | Preproenkephalin |
| 24 | −1.57 | Y | G | G | F | L | K | V00510/J00123 |
| | | | | | | | | Preproenkephalin |
| 33 | −1.65 | Y | G | G | F | M | T | M25896 gamma-endorphin |

It is important to note that this approach can be used to identify ligands within proteins in databases for any molecular interaction that has been or can be studied with PS-SCL composed of not only L-amino acids, but also other strings of similar building blocks, e.g., sugar molecules, lipid molecules, nucleotides, D-amino acids and other synthetic derivatives and compounds that can be employed following similar principles, i.e., additive and independent contribution of each building block to the interaction. Furthermore, the length of such molecules and libraries is not limited to decamers. Thus, the integration of data derived from PS-SCL screening with biometrical analysis can be applied to biological targets having peptides as natural ligands, such as T cells, proteolytic enzymes and cellular signal receptors. Such interactions include those between a ligand and antibodies, MHCs, nucleic acids, amino acids, peptides, intracellular and extracellular enzymes, intracellular and extracellular gated channels, cellular signal receptors, and any other molecular interaction that elicits a functional response. A ligand is an epitope, hormone, growth factor, promoter, repressor, cofactor, antigen, amino acid, ion, cellular signal, therapeutic agent, or any molecule that binds specifically to a receptor molecule. A functional response includes binding, conformational modification, enzymatic activity, promotion or repression, cleavage, transport, signal transduction, growth, proliferation, necrosis, and apoptosis.

### EXAMPLE 1

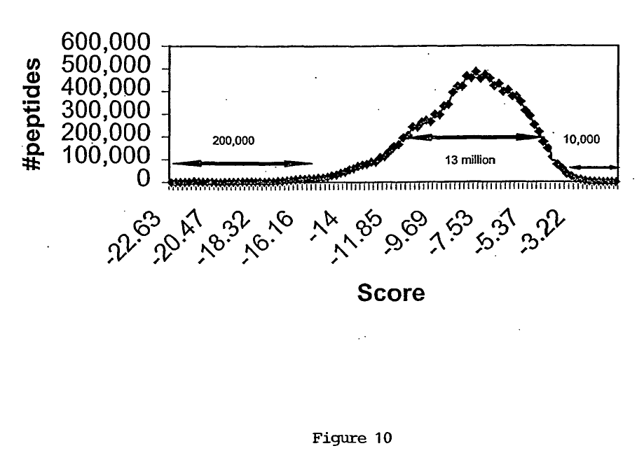

Referring to FIG. 11, a system 10 for determining proteins that would stimulate a particular T cell is disclosed. The system 10 includes a main system 20 that in one embodiment is a Microsoft® Windows-based personal computer system and in another embodiment is a Unix-based platform.

As illustrated, a set of combinatorial library stimulation data 25 is used to determine a positional scoring matrix 30. Then, an analysis module 35 runs instructions in the system 20 to search a protein database 50 for epitopes that are anticipated to stimulate the chosen T cell based on the data within the positional scoring matrix 30. The protein database in one embodiment is the GenPept (http://www.ncbi.nlm.nih.gov/entrez/) database, in another embodiment is the Protein Information Resource (http://helix.nia.gov/science/pir.html) database, and in another embodiment is the SWISSPROT (http://www.expasy.ch/sprot/) database.

During the search through the protein database 50, the analysis module 35 uses the positional scoring matrix 30 to scan, for example, every unique ten-amino-acid sequence within every protein in the protein database 50 to search for those epitopes that, during training, were found to stimulate the chosen T cell. Once such proteins are identified within the protein database 50, an output list 60 is created of all proteins within the protein database that have been determined to stimulate the chosen T cell.

A sample of high-scoring peptides are synthesized and experimentally tested. The stimulation data 64 for the tested peptides, in conjunction with the scoring matrix, are used to train an ANN module 66 for performing a neural network analysis to recognize patterns of epitopes that stimulate the chosen T cell. Accordingly, a refined list of peptides 68 that stimulate the chosen T cell is determined from the neural network analysis.

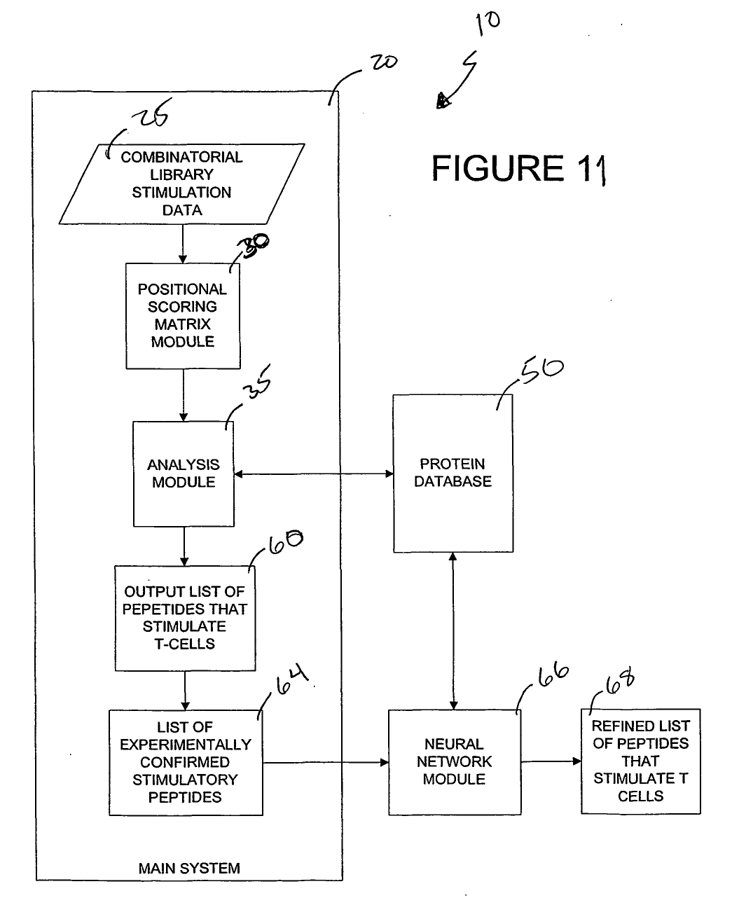

Thus, the system 10 provides an efficient mechanism for determining proteins in a protein database that would stimulate a particular T cell. This allows investigators to rapidly determine known proteins that may trigger, for example, autoimmune diseases. The process of analyzing protein epitopes and outputting a list of ones that stimulate T cells is explained more completely in FIG. 12.

Referring now to FIG. 12, a process 100 of ranking and determining proteins that stimulate a particular T cell is explained. The process 100 begins at a start state 102 and then moves to a state 104 wherein the main system 20 receives individual data on the stimulatory effect of a T cell by a panel of combinatorial peptide libraries. The process 100 then moves to a state 106 wherein a positional scoring data matrix 40 is calculated. This process is explained below.

In order to create a positional scoring matrix, each of the 200 peptide libraries representing all possible 10 amino acid epitopes are experimentally tested against a particular T cell clone. So as to provide higher accuracy, replicate measurements of the stimulation value of the T cell clone for each combinatorial library are taken. In the data preprocessing step, a locally weighted regression smoothing technique (S-plus package, version 4.5) is used to derive a smoothed estimate of the standard deviation of each measurement based on the assumption that the standard deviation is dependant on level of response by the T cell.

A positional scoring matrix is then generated by assigning a value of the stimulatory potential to each of 20 amino acids in each position. The score S, for each amino acid i at each position j is calculated as follows:

$${S}_{i j} = \frac{{L}_{i j} - B}{\sqrt{{( \text{std} ( {L}_{i j} ) )}^{2} + {( std ( B ) )}^{2}}}$$

Where L equals the mean of replicate experimental measurements (Counts per minute or % target cell lysis), B is background noise, std(Lᵢⱼ) denotes the smoothed estimate of the standard deviation for each measurement.

Under the assumption that each amino acid provides an independent contribution to stimulation, the predicted stimulatory potential of a given peptide is the sum of the scores in each position. A peptide with 10 amino acids can be represented by a 20×10 matrix of 0's and 1's (pᵢⱼ) where pᵢⱼ=1 if the i th amino acid (using the same order as for the rows of the scoring matrix) is in position j. Let S, denote the components of the positional scoring matrix. Then the score for the peptide is

$$S = {\sum }_{i = 1}^{20} {\sum }_{j = 1}^{10} {p}_{i j} {S}_{i j}$$

A statistical significance test of the hypothesis that the score for a peptide is no greater than would be expected if the peptide were obtained from 10 random draws of amino acids was developed. Under the null hypothesis it is not assumed that all amino acids are equally likely, but rather the relative frequencies f₁, f₂, . . . , f₂₀ are derived from the database being searched. Under the null hypothesis, the distribution of S will be approximately normally distributed. The mean and the variance of this null distribution can be expressed as

$$\begin{matrix}m = {\sum }_{i = 1}^{20} {f}_{i} {\sum }_{j = 1}^{10} {S}_{i j} \\ var = E [ {S}^{2} ] - {m}^{2}\end{matrix}$$

The variance can be shown to equal

$$\begin{matrix}var = {\sum }_{i = 1}^{20} {f}_{i} {\sum }_{j = 1}^{10} {S}_{i j}^{2} + 2 {\sum }_{j = 1}^{9} {\sum }_{{j}^{′} = j + 1}^{10} {m}_{j} {m}_{{j}^{′}} - {m}^{2} \\ where {m}_{j} = {\sum }_{j = 1}^{20} {f}_{ i} {S}_{i j} .\end{matrix}$$

The statistical significance of any score S can be approximated as:

$$p = Φ ( \frac{m - S}{\sqrt{var}} )$$

where Φ denotes the standard normal distribution function. This significance level does not, however, account for the number of 10 amino acid sequences contained in the database.

Once the scoring matrix has been determined at the state 106, a database of proteins to be tested is accessed at a state 110. The process 100 then moves to a state 112 wherein the first protein within the database is selected. At a state 114, the first ten amino acids corresponding to a single epitope of the protein is selected. The process 100 then moves to a state 116 wherein the ten-amino-acid epitope is evaluated using the scoring matrix. The stimulation potential for the selected ten amino acids of the protein is then determined at a state 120. The process 100 then moves to a state 124 wherein the stimulation potential of the ten-amino-acid epitope in the protein is stored to a database.

A determination is then made at a decision state 126 whether more amino acids are available in the protein to be analyzed. As can be envisioned, the process 100 scans every ten-amino-acid sequence within the protein in order to determine which epitopes stimulate the chosen T cell. If a determination is made that no more amino acids are available for analysis in the protein, the process 100 moves to a decision state 130 to determine whether more proteins are available in the database. If no more proteins are available, the process 100 moves to a state 134 wherein each protein is ranked by the stimulatory potential of the epitopes within the protein.

Once the initial stimulatory potential of the epitopes is determined, a sample of such peptides are synthesized and experimentally tested for the ability to bind the T cell receptor in the MHC-restricted manner at a state 135. Using that data, data for any other peptides are experimentally tested against the T cell clone and the scoring matrix, and the ANN 30 is trained at a state 136 in order to learn which amino acids within particular positions of the epitope are necessary for T cell stimulation.

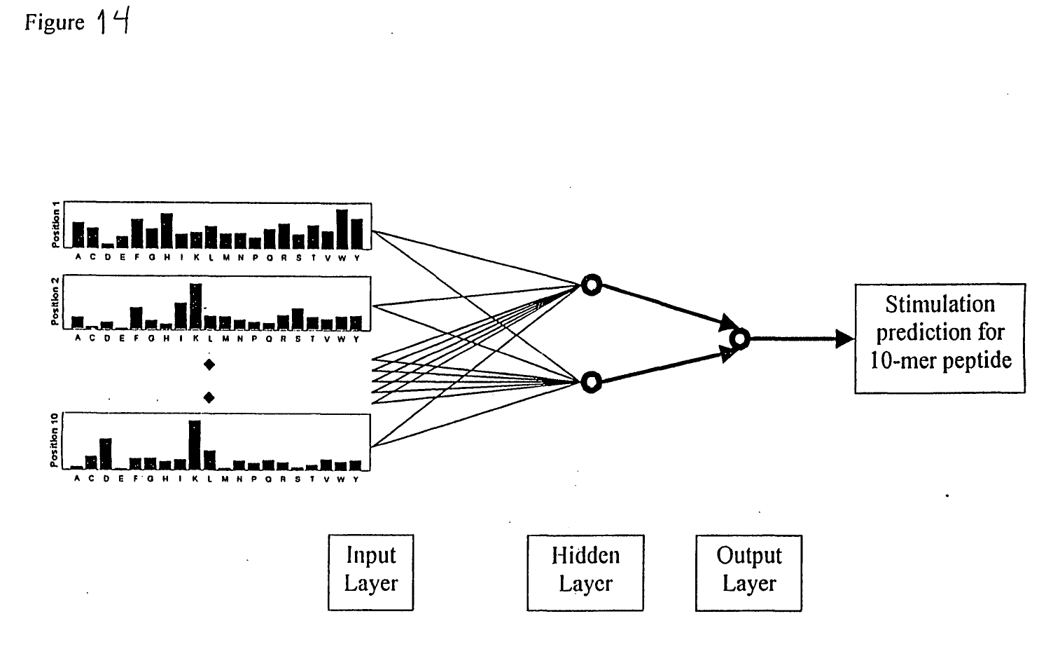

The ANN analysis is performed using in one embodiment the Neuroshell 2 software package (Ward Systems Group). However, it should be realized that any similar program would function within the scope of the invention. A feed-forward, error-back-propagation architecture is chosen with three layers (single hidden layer). There are ten neurons in the input layer, which represent the ten-amino-acid positions in the peptide. A diagram of one examplary ANN is illustrated in FIG. 14 and discussed more thoroughly below.

The T cell stimulation scores Sᵢⱼ of each amino acid in each position were used as input values to the ANN. There are two neurons in the hidden layer. The output values were set to 1 for stimulatory peptides and 0 for non-stimulatory peptides. Since the percentages of positive and negative peptides were very different (28:116), the data was first divided into positive and negative groups. Random samplings in each group were then used to give a training set (70% of the total peptides), a test set (10% of the total peptides), and a production set (20% of the total peptides). Finally, the positive and negative groups were combined separately in the training, test, and production sets.

The threshold value for predicting whether the T cell would be stimulated or non-stimulated by the peptide was set to 0.5 for the ANN analysis. The weights of the ANN were fit using data in the training set. The ANN fitting stopped when there was no error decrease in the test set. The training was repeated five times with random drawings using different seeds. The final indexes were the average of all training results.

Once the final indexes were calculated, peptides that are predicted to have the most stimulatory effect on T cells are determined at a state 138. The process then terminates at an end state 140.

It should be realized that if a determination is made at the decision state 126 that more amino acids were available for analysis in the protein, the process 100 moves to a state 142 wherein the next ten-amino-acid sequence is selected. Normally, the next ten-amino-acid sequence is selected by moving a scanning window of ten amino acids to the right by one amino acid in the protein. Thus, overlapping windows of ten-amino-acid epitopes are analyzed in the protein.

If a determination is made that more proteins did exist in the database at the decision state 130, the process 100 moves to a state 144 wherein the next protein is retrieved from the database. The process then returns to the state 114 wherein the first ten amino acids of the newly selected protein are selected.

In one embodiment, a Perl script was written to systematically search the GenPept database (release 108) and retrieve potential binding peptides. A window with the same number of amino acids as used in creating the PS-SCL matrix was applied to scan over the 334,216 translated protein-coding sequences. The sum of the scores within the window was used as a ranking criterion. All peptides with scores higher than a threshold value were sent to the output file 60. The threshold value was chosen based on the statistical significance of the peptide score, compared to that for a random peptide. Those peptides were then sorted. Redundant peptides were removed. A keyword search of the sequence annotation was used to select human or viral sequences during the database search.

TL3A6 is a DRB5*0101-restricted T cell clone that was obtained from a multiple sclerosis (MS) patient that had been generated by stimulation with myelin basic protein (MBP) (Hemmer et al., J Exp Med, 185:1651-1659). TL3A6 is specific for the immunodominant MBP peptide having the amino acid sequence VHFFKNIVTPRTP (SEQ ID NO:4). This TCC was tested for its response to completely randomized peptide libraries.

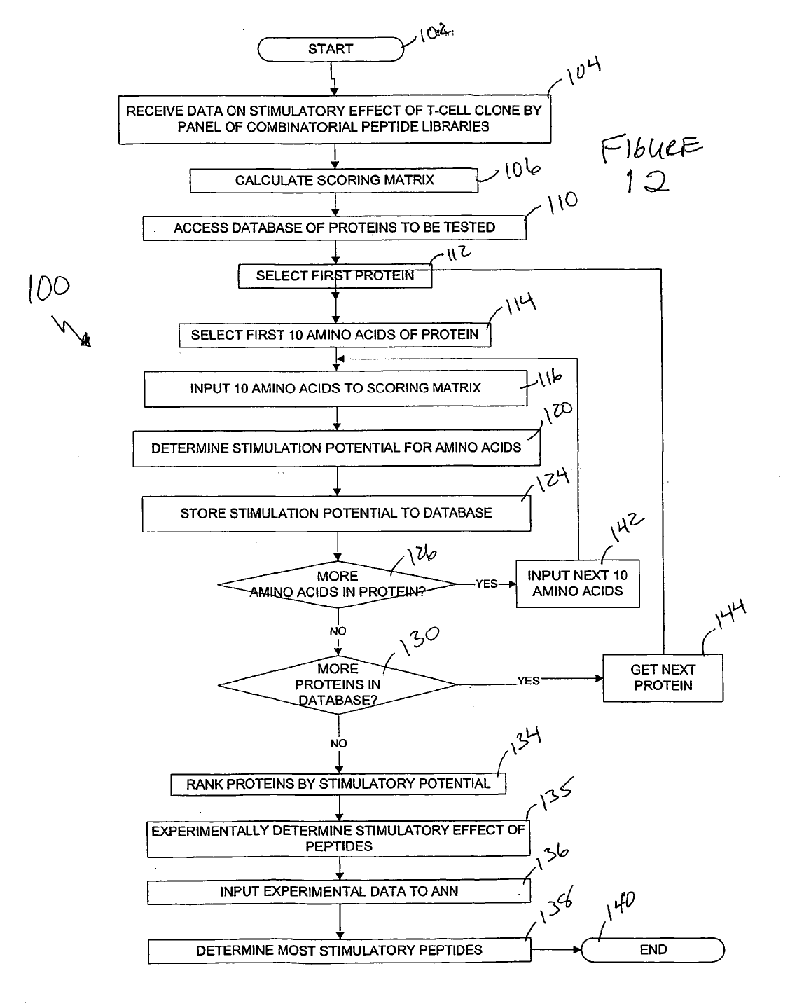

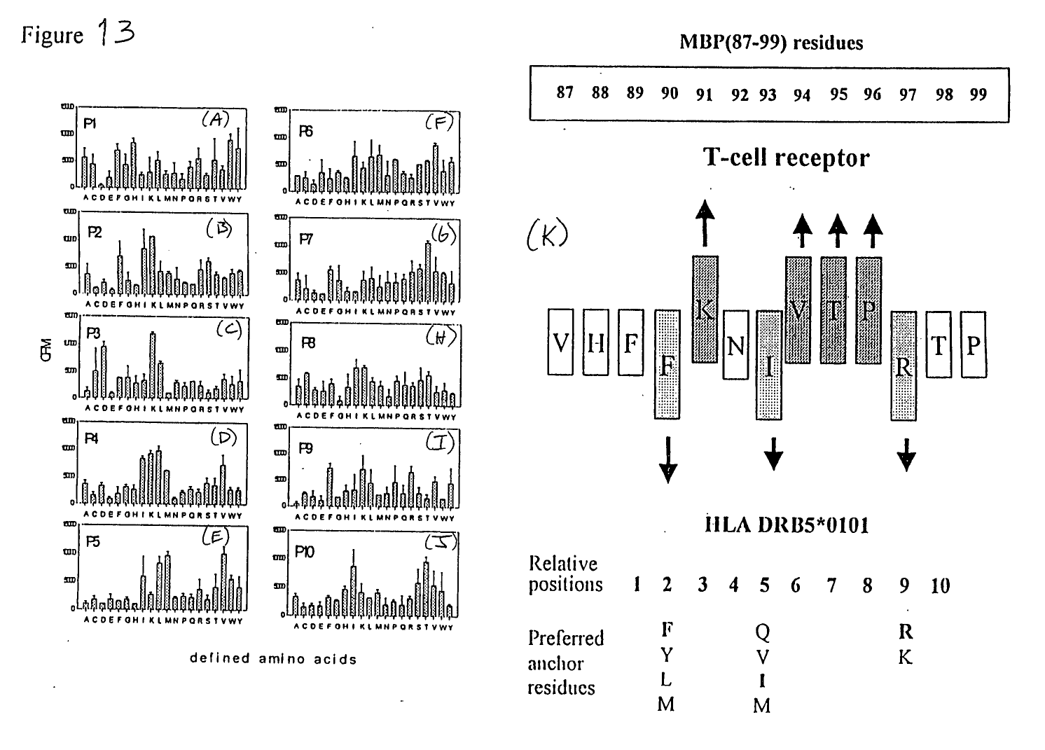

A total of 200 sublibraries were used. In each sublibrary, only one amino acid was fixed in one position while the other 9 positions were each randomized with a mixture of all 20 L-amino acids except cysteine (to avoid formation of secondary structures). Thus, 10×20=200 sublibraries were produced. FIGS. 13A-J show the proliferative response of T cell clone TL3A6 to each of the 200 10 amino acid sublibraries. The result of the PS-SCL experiment reflects not only the primary anchor residues for HLA-DRB5*0101 binding, but also, at the same time, delineates those residues that are important for contacting the T cell receptor (FIG. 13K) from those residues that are not important.

An “integration” model was used wherein the combination of positive and negative effects of individual amino acids in the antigenic peptide determines whether the resulting affinity of the MHC/peptide ligand for T cell receptor is high enough to trigger T cell receptor-dependent signaling events. Although structural studies have demonstrated that amino acid side chains of the peptide that contact either T cell receptor or MHC molecules contribute disproportionately to recognition, and although certain influences between adjacent amino acids exist, the assumption of independent contributions of side chains is a reasonable approximation of T cell stimulation and provides a good starting point for building a mathematical model.

Based on the above assumptions, and using data obtained from testing T-cell clones (TCC) with PS-SCL, a scoring matrix (Table A) was calculated which assigns numerical values for the stimulatory potential of each amino acid in each position, as described below. Using the scoring matrix, the stimulatory potential of a peptide is calculated by summing the scores assigned to each amino acid in each position of the peptide. For a 10-mer PS-SCL experiment, for example, the GenPept protein database was searched by moving a 10-mer window along all sequences in the database and scoring all 10-mer peptides. All high scoring 10-mers were thereby identified. For example, the score of the target segment FFKNIVTPRT (SEQ ID NO:2) ranks 225th among 31,129 human proteins with a p-value of 5.3×10⁻⁷, thus also confirming that the autoantigen is far from an optimal ligand for TCC TL 3A6.

TABLE A
Score matrix for TL3A6 clone.

| | Position 1 | Position 2 | Position 3 | Position 4 | Position 5 | Position 6 | Position 7 | Position 8 | Position 9 | Position 10 |
|---|---|---|---|---|---|---|---|---|---|---|
| A | 2.85 | 1.47 | −0.30 | 1.52 | −0.54 | 1.02 | 1.54 | 1.40 | −1.06 | 1.34 |
| C | 1.84 | −0.52 | 2.07 | 0.02 | 0.14 | 0.81 | 0.45 | 3.09 | 0.76 | −0.11 |
| D | −1.12 | 0.48 | 6.28 | 1.34 | −0.58 | −0.25 | −0.27 | 0.97 | 0.21 | 0.17 |
| E | 0.32 | −0.86 | −0.72 | −0.55 | 0.22 | 1.42 | −0.44 | 0.86 | −0.49 | 0.10 |
| F | 3.59 | 3.58 | 1.70 | 0.26 | −0.14 | 0.76 | 2.83 | 1.77 | 3.65 | 1.24 |
| G | 1.83 | 0.80 | 1.74 | 1.21 | 0.21 | 1.46 | 1.58 | −0.81 | 0.17 | 0.87 |
| H | 4.60 | 0.06 | 1.03 | 0.90 | −0.58 | 0.82 | 0.09 | 1.34 | 1.07 | 1.91 |
| I | 0.79 | 4.55 | 1.39 | 4.50 | 3.16 | 3.55 | 0.00 | 3.58 | 1.24 | 5.03 |
| K | 1.11 | 8.55 | 10.49 | 5.73 | 0.91 | 1.85 | 1.60 | 3.58 | 3.59 | 1.79 |
| L | 2.23 | 1.82 | 3.53 | 6.71 | 4.34 | 3.50 | 1.79 | 1.86 | 1.86 | 1.20 |
| M | 0.90 | 1.72 | −0.57 | 3.33 | 6.55 | 3.57 | 0.80 | 1.48 | 0.62 | 1.77 |
| N | 1.00 | 0.99 | 1.17 | −0.49 | 0.47 | 1.15 | 1.39 | 0.04 | 0.82 | 0.32 |
| P | 0.16 | 0.54 | 0.68 | 0.57 | 0.72 | 3.22 | 1.39 | 1.89 | 1.93 | 0.81 |
| Q | 1.77 | 0.35 | 1.34 | 0.94 | 0.57 | 1.40 | 1.77 | 1.66 | 0.83 | 0.33 |
| R | 2.80 | 1.94 | 0.81 | 0.59 | 1.55 | 0.89 | 2.41 | 1.47 | 3.54 | 1.16 |
| S | 0.82 | 3.37 | −0.40 | 1.74 | 0.26 | 2.12 | 3.24 | 1.95 | 0.89 | 3.12 |
| T | 2.45 | 1.61 | 0.26 | 1.39 | 1.77 | 2.98 | 8.42 | 2.87 | 0.04 | 6.37 |
| V | 1.43 | 1.20 | 1.57 | 3.64 | 7.30 | 4.94 | 2.64 | 0.81 | 1.98 | 2.52 |
| W | 5.60 | 1.77 | 0.99 | 0.92 | 2.67 | 1.75 | 2.14 | 1.05 | 0.02 | 1.86 |
| Y | 3.84 | 1.87 | 1.37 | 0.91 | 1.78 | 2.89 | 1.36 | 0.65 | 1.87 | 0.30 |

Data from a PS-SCL experiment with TL3A6 clone was used to generate this score matrix. Each amino acid in each position is assigned a stimulatory potential. The score of a decapeptide is determined by summing the values of the individual amino acids from the score matrix. The amino acids are indicated with one-letter code.

144 synthetic peptides were synthesized and tested for stimulation of the TL3A6 clone based on single- and multiple-amino acid-substitutions and PS-SCL experiments (Hemmer et al., J Immunol, 160:3631-3636). To establish the validity of our assumptions experimentally, we compared experimental data and calculated stimulatory scores for each of the peptides. A peptide with an EC50 value one order of magnitude lower than the target MBP segment was defined as positive. The EC50 is an estimate of the peptide concentration needed to achieve half maximal proliferation (counts per minute; cpm). Of 144 synthetic peptides, 28 were positive and 116 were negative.

To evaluate the adequacy of our predictions, five indexes were used: sensitivity, specificity, positive-predictive value (PPV), negative-predictive value (NPV), and accuracy. After performing a Relative Operating Characteristic (ROC) analysis (Swets, J. A., Science, 240:1285-1293), a threshold score was chosen for the binary prediction. A peptide was predicted to be stimulatory if its score exceeded this threshold. The classification of the 144 synthesized peptides using the scoring matrix was developed from the PS-SCL data. The overall accuracy was 82%, however, the PPV was relatively poor, which indicates that the assumption of independent amino acid contribution was likely violated and that some interactions probably existed among the amino acid residues in the peptide.

In order to attempt to improve on the positional scoring matrix model, we developed an ANN to predict the ability of a peptide to stimulate a T cell. A full ANN with an indicator for each amino acid at each position would require 200 input nodes (20×10). Large ANNs require very large amounts of data to avoid obtaining poor predictions resulting from over-fitting a limited set of training data. The number of weights for edges joining m input nodes to h hidden layers is hm. Hence even with only h=1, a prohibitive amount of data is required for properly training a network with m=200.

For this reason, we used one input node per amino acid position in the peptide, and employed the positional scoring matrix entries for the peptide as input values. This limited the number of input nodes to 10 (one for each position) and still provided model flexibility for identifying non-linearities in the data. We then applied the ANN analysis to the 144 synthesized peptides of the TL3A6 clone.

FIG. 14 shows the basic architecture of the ANN used in these experiments. The whole data set was divided into three parts: Training, test, and production sets. The training set consists of input variables and correct output variables used in training the neural network. The test set was used to optimize the network parameters during training. The production set was used to test, in an unbiased way, how well the network was performing. ANN training greatly improved the PPV percentage, which, as discussed above, was poor for the positional scoring matrix, and also improved the accuracy by 10 percentage points.

In order to understand the ANN model better, a statistical method of tree-based modeling was applied to the data in order to improve upon the accuracy of the positional scoring matrix. Decision tree modeling provided an alternative to linear and additive logistic models for classification problems. The rules were determined by a procedure known as recursive partitioning (Stryhn et al., Eur J Immunol, 26:1911-1918), which allows more general interactions between different predictor variables.

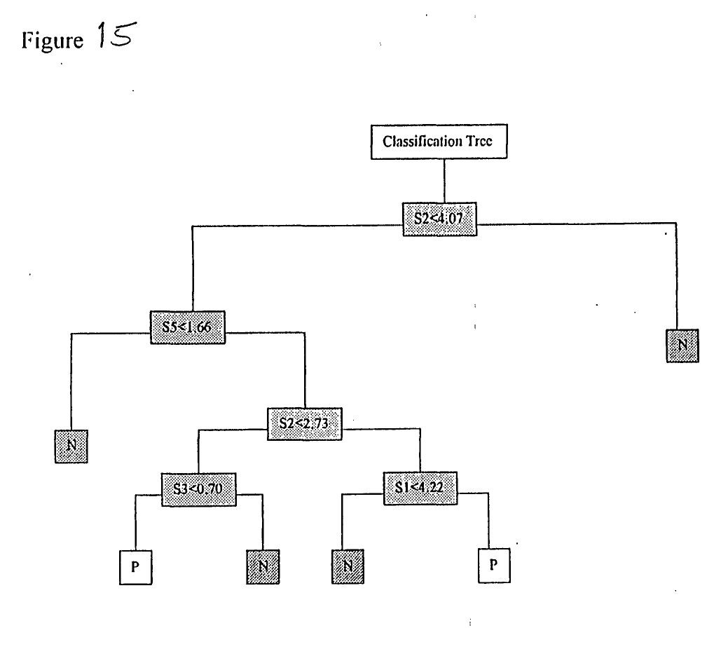

The same 10 variables (one for each amino acid position of the ligand) that were used as input nodes for the ANN were used for the decision tree. The classification tree was built using the data set of the TL3A6 clone. The decision tree provided almost the same performance as the ANN. The typical classification tree (FIG. 15) identified several important interactions between MHC anchor sites and T cell receptor binding sites. If the score of amino acid in that position is greater than the number indicated in the box, the tree will go right, otherwise, go left. The letters ‘P’ and ‘N’ in the lower boxes stand for positive (stimulatory) and negative (non-stimulatory) results. In this example, combinations of amino acids in position 2 and 5; position 2, 5, and 3; and position 2, 5, 1 gave positive (stimulatory) or negative (non-stimulatory) peptides.

While embodiments of this invention have been described using ANN analysis to determine binding epitopes of T cells, the invention is not limited. Other mathematical models of predicting T cell stimulation are also anticipated. For example, classification trees can be used to determine binding epitopes through analysis of the positional scoring data.

A classification tree identifies which variable to use for a binary division of the cases at that node and what threshold to use for the division. Two edges leave each node. These edges are viewed as separate paths for the cases divided into two groups at the node. The two subsets of cases determined by the division at a node are separately analyzed and sub-divided at subsequent nodes. All cases reach some terminal node. Each terminal node is classified based on the plurality class at that node.

A classification tree was generated using the S-plus 4.5 software package. The scores Sᵢⱼ of each amino acid in each position were used as numerical predictors. The responses were set to 1 for stimulatory peptides and 0 for non-stimulatory peptides. The data sets developed for the neural networks were used. The training set (70% of the total peptides) was the same as the training set for neural network. The test set (20% of the total peptides) was the production set in neural network. The process was repeated five times with different random drawings. The final indexes were the average of all training results.

| Sequence | SEQ ID NO: |
|---|---|
| YVKQNTLKLA | SEQ ID NO: 1 |
| FFKMVTPRT | SEQ ID NO: 2 |
| PKYVKQNTLKLAT | SEQ ID NO: 3 |
| VHFFKNIVTPRTP | SEQ ID NO: 4 |
| WYFRHMLIVADFYHKQVILYHKPTMNHQMTSNIQGVA | SEQ ID NO: 5 |
| HMGPAHFSTYVNQLICMRKGPMTNVSFRMYKLVHQPN | |
| ISWGALMIFVYQA | |
| WYFRMLIVADFKQVILYHKPNHQTSNIQGVAHGPAHF | SEQ ID NO: 6 |
| STYVNQLRKGPMFRMYKLVHQPNILMIFVY | |
| WHYFARTLCGQVKNKIFSRYLWMTAVNKDLCGFVIYQ | SEQ ID NO: 7 |
| NHLKIMVSATDGVMLIWYTRVMILPTYSKWGEQNATS | |
| FVRWLQKGAPNYKICTSPLFQMRAHWFKRVPYLIHTI | |
| SVHWKMAFLR | |
| WHYFARTLCGKIFSRYLKDLCLKIMVVMLIWVMILPT | SEQ ID NO: 8 |
| YSKTSFVRWKICTSPLFKRVPYLTISVHW | |
| WMKQNIGRFL | SEQ ID NO: 9 |
| NNIYKKALIS | SEQ ID NO: 10 |
| SNIIKSLSLF | SEQ ID NO: 11 |
| SNIIKKTSED | SEQ ID NO: 12 |
| FNIYKRVVDN | SEQ ID NO: 13 |
| NNIDKKVYTN | SEQ ID NO: 14 |
| FFIKKRSLII | SEQ ID NO: 15 |
| RNIFKKTVEN | SEQ ID NO: 16 |
| SNIKSKLILV | SEQ ID NO: 17 |
| YNIIVSSLLL | SEQ ID NO: 18 |
| DNIFKKETLI | SEQ ID NO: 19 |
| QAIGKKIQNN | SEQ ID NO: 20 |
| TLITKKISAI | SEQ ID NO: 21 |
| LNIKNSKLEI | SEQ ID NO: 22 |
| FNIIKVHSSL | SEQ ID NO: 23 |
| YNIKKIKVED | SEQ ID NO: 24 |
| LNITSSSYLF | SEQ ID NO: 25 |
| ENIKKILLRE | SEQ ID NO: 26 |
| NNIKSKVDNA | SEQ ID NO: 27 |
| YSICKSGCFY | SEQ ID NO: 28 |
| LHIISKRVEA | SEQ ID NO: 29 |
| SFIYSVVCLV | SEQ ID NO: 30 |
| GHIKKKRVEA | SEQ ID NO: 31 |
| FNITSSTCEL | SEQ ID NO: 32 |
| ENVKKSRRLI | SEQ ID NO: 33 |
| DNITSSVLFN | SEQ ID NO: 34 |
| FNIIKSLLGG | SEQ ID NO: 35 |
| PNITFSVVYN | SEQ ID NO: 36 |
| FNITSSIRNK | SEQ ID NO: 37 |
| ENIYYSSVRT | SEQ ID NO: 38 |
| YGGFMR | SEQ ID NO: 39 |
| YGGFLR | SEQ ID NO: 40 |
| YGGFMK | SEQ ID NO: 41 |
| YGGFLK | SEQ ID NO: 42 |
| YGGFMT | SEQ ID NO: 43 |
| YIKQNTLKLS | SEQ ID NO: 44 |
| YIDDNSKKVF | SEQ ID NO: 45 |
| EPASAKEWDR | SEQ ID NO: 46 |
| SLYFCASS | SEQ ID NO: 47 |
| MPGQGGG | SEQ ID NO: 48 |
| TDTQYFGPGTRLTVL | SEQ ID NO: 49 |
| EDLK | SEQ ID NO: 50 |

### REFERENCES

1. Alekshun, M., Kashlev, M. & Schwartz, I. Molecular cloning and characterization of Borrelia burgdorferi rpoB. Gene 186, 227-235 (1997).

2. Bono, J. L., Tilly, K., Stevenson, B., Hogan, D. & Rosa, P. Oligopeptide permease in Borrelia burgdorferi: putative peptide-binding components encoded by both chromosomal and plasmid loci. Microbiology 144, 1033-1044 (1998).

3. Derynck, R. et al. A new type of transforming growth factor-beta, TGF-beta 3. EMBO J. 7, 3737-3743 (1988).

4. Fraser, C. M. et al. Genomic sequence of a Lyme disease spirochaete, Borrelia burgdorferi. Nature 390, 580-586 (1997).

5. Gao, F. et al. Molecular cloning and analysis of functional envelope genes from human immunodeficiency virus type 1 sequence subtypes A through G. The WHO and NIAID networks for HIV isolation and characterization. J. Virol. 70, 1651-1667 (1996).

6. Labeit, S., B. Kolmerer. 1995. Titins: giant proteins in charge of muscle ultrastructure and elasticity. Science 270:293-296.

7. Lam, T. T., Nguyen, T. P., Fikrig, E. & Flavell, R. A. A chromosomal Borrelia burgdorferi gene encodes a 22-kilodalton lipoprotein, P22, that is serologically recognized in Lyme disease. J. Clin. Microbiol. 32, 876-883 (1994).

8. Li, L., Wang, J. & Cooper, M. D. cDNA cloning and expression of human glutamyl aminopeptidase (aminopeptidase A). Genomics 17, 657-664 (1993).

9. Nanus, D. M. et al. Molecular cloning of the human kidney differentiation antigen gp160: human aminopeptidase A. Proc. Natl. Acad. Sci. USA 90, 7069-7073 (1993).

10. Old, I. G., Margarita, D. & Saint Girons, I. Unique genetic arrangement in the dnaA region of the Borrelia burgdorferi linear chromosome: nucleotide sequence of the dnaA gene. FEMS Microbiol. Lett. 111, 109-114 (1993).

11. Padula, S. J., Sampieri, A., Dias, F., Szczepanski, A. & Ryan, R. W. Molecular characterization and expression of p23 (OspC) from a North American strain of Borrelia burgdorferi. Infect. Immun. 61, 5097-5105 (1993).

12. Pieniazek, N. J., Slemenda, S. B., Pieniazek, D., Velarde, J., Jr. & Luftig, R. B. Human enteric adenovirus type 41 (Tak) contains a second fiber protein gene. Nucleic Acids Res. 18, 1901 (1990).

13. Sallusto, F. et al. Rapid and coordinated switch in chemokine receptor expression during dendritic cell maturation. Eur. J. Immunol. 28, 2760-2769 (1998).

14. Schweickart, V. L. et al. Cloning of human and mouse EBI1, a lymphoid-specific G-protein-coupled receptor encoded on human chromosome 17q12-q21.2. Genomics 23, 643-650 (1994).

15. Sims, J. E. et al. Cloning the interleukin 1 receptor from human T-cells. Proc. Natl. Acad. Sci. USA 86, 8946-8950 (1989).

16. Sprenger, H. et al. Borrelia burgdorferi induces chemokines in human monocytes. Infect. Immun. 65, 4384-4388 (1997).

17. Thomson, B. J., Efstathiou, S. & Honess, R. W. Acquisition of the human adeno-associated virus type-2 rep gene by human herpesvirus type-6. Nature 351, 78-80 (1991).

18. Trueba, G. A., Old, I. G., Saint Girons, I. & Johnson, R. C. A cheA cheW operon in Borrelia burgdorferi, the agent of Lyme disease. Res. Microbiol. 148, 191-200 (1997).

19. Yamada, Y. et al. Cloning and functional characterization of a family of human and mouse somatostatin receptors expressed in brain, gastrointestinal tract, and kidney. Proc. Natl. Acad. Sci. USA 89, 251-255 (1992).

20. Yamamoto, Y. et al. Cloning and expression of myelin-associated oligodendrocytic basic protein. A novel basic protein constituting the central nervous system myelin. J. Biol. Chem. 269, 31725-31730 (1994).

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

What is claimed is:

1. A method of determining the amino acid sequence of a peptide that stimulates a functional response, comprising:
identifying at least a first and a second amino acid position of the peptide;
defining a stimulating potency level of each of a plurality of amino acids within the first position of the peptide;
defining a stimulatory potency level of each of a plurality of amino acids within the second position of the peptide; and
evaluating selected positions of a protein with the at least first and second positions of the peptide to determine the stimulatory potency level of amino acids in said selected positions.

2. The method of claim 1 wherein the acts of defining a potency level are performed for all amino acids within the first and second positions of the peptide.

3. The method of claim 1, further comprising:
assigning score values to the determined stimulatory potency levels for a first group of the selected positions; and
developing a first composite score representing the stimulatory potency level of a first group of positions in the protein corresponding to the positions in the peptide.

4. The method of claim 3, further comprising:
developing a second composite score representing the stimulatory potency level of a second group of positions in the protein corresponding to the positions in the peptide; and
selecting from among the first and second composite score the score identifying the group of positions in the protein having the greater stimulatory potency level.

5. The method of claim 4, wherein the first and second composite scores are the sum of the score values of the determined stimulatory potency levels of the respective first and second groups.

6. The method of claim 1, wherein the determined amino acid sequence is that of a peptide epitope that stimulates a T cell.

7. The method of claim 1, wherein the act of defining a stimulatory potency level comprises testing each amino acid within the at least a first and second position of the peptide to determine its stimulatory potency; and
assigning a score to each amino acid representing the level of its stimulatory potency.

8. A method of determining the amino acid sequence of a peptide that stimulates a functional response, comprising
identifying a position of the peptide;
testing each amino acid on the identified position so as to define a stimulatory potency level for each of said amino acids; and
selecting one of the amino acids, based upon its defined stimulatory potency level, as the amino acid in said position which produces the greatest stimulatory potency.

9. The method of claim 8 wherein the act of identifying a position comprises identifying a selected number of positions and wherein the acts of testing each amino acid and selecting one amino acid are repeated for each of the selected number of identified positions.

10. The method of claim 8, wherein the selected number of positions is ten positions.

11. A method of determining the amino acid sequence of a peptide that stimulates a functional response comprising:
identifying a selected number of positions of the peptide; and
testing each amino acid on each identified position so as to define a stimulatory potency level for each of said amino acids.

12. The method of claim 11 farther comprising:
evaluating a first group of said selected number of positions of a protein, with the selected number of positions of the peptide to determine the stimulatory potency level of amino acids in the positions of the first group.

13. The method of claim 12 further comprising:
assigning score values to the determined stimulatory potency levels for each of the positions of the first group; and
developing a first composite score representing the stimulatory potency level of the first group of positions in the protein.

14. The method of claim 13 further comprising:
evaluating a second group of said selected number of positions of the protein, with the selected number of positions of the peptide to determine the stimulatory potency level of amino acids in the positions of the second group;
assigning score values to the determined stimulatory potency levels for each of the positions of the second group;
developing a second composite score representing the stimulatory potency level of the second group of positions in the protein; and
selecting from among the first and second composite scores the score identifying the group of positions in the protein having the greater stimulatory potency level.

15. The method of claim 14, wherein the first and second composite scores are the sum of the score values of the determined stimulatory potency levels of the respective first and second groups.

16. The method of claim 12 wherein the act of evaluating comprises:
comparing amino acids of a position in the peptide with the amino acid of the corresponding position of the protein to identify the amino acid of the position of the protein; and
assigning the value of the corresponding amino acid in the corresponding position of the peptide to the identical amino acid.

17. The method of claim 11, herein the first and second composite scores are the sum of the score values of the determined stimulatory potency levels of the respective first and second groups.

18. The method of claim 11, wherein the determined amino acid sequence is that of a peptide epitope that stimulates a T cell.

19. A method of scoring the ability of amino acids within a position on a peptide to stimulate a functional response, comprising:
conducting a plurality of measurements of the stimulation value of each of said amino acids within a position on the peptide;
identifying a mean value (L) for said measurements;
identifying a value for background noise in said measurements (B); and
identifying a smoothed estimate of a standard deviation std(L) and std(13) for each of said measurements;
determining a score for each of the i amino acids within said position using the relationship:
$${S}_{i} = \frac{{L}_{i} - B}{\sqrt{{( std ( {L}_{i} ) )}^{2} + {( std ( B ) )}^{2}}}$$

20. The method of claim 19 further comprising repeating said method for each of the amino acids in each of plural positions in the peptide.

21. The method of claim 20, further comprising storing the score values in a matrix configured with the amino acids defining one axis thereof and the peptide positions defining the other axis.

22. A method of evaluating a protein to identify a peptide having a desired level of stimulatory potency, comprising:
providing a peptide having a defined number of positions;
scoring the stimulatory potency of each plurality of amino acids in each plurality of the positions with respect to a functional response;
defining a template including the scores of the amino acids in each of the positions;
applying the scores of the template to amino acids in corresponding positions on selected portions of the protein;
summing amino acid scores at each of a plurality of portions of the protein, to produce a stimulatory potency score for each of said portions; and
comparing the stimulatory potency scores for each of said portions so as to identify a portion of the protein having a desired level of stimulatory potency.

23. The method of claim 22, wherein the act of scoring the stimulatory potency of a plurality of amino acids comprises individually scoring the stimulatory potency of all amino acids in all of the plurality of positions.

24. The method of claim 23 wherein the plurality of positions comprises ten positions.

25. The method of claim 22 wherein the act of defining a template comprises defining a matrix configured such that one axis of the matrix comprises each of the amino acids and the other axis comprises each of the plurality of positions of the peptide.

26. The method of claim 22, wherein the act of comparing the stimulatory potency scores comprises identifying a portion of the protein having the highest score from among those compared.

27. The method of claim 22 wherein the act of applying the scores comprises:
selecting a first group of positions of the protein corresponding in number to the number of positions in the template; and
applying the scores for the amino acid in each position of the peptide that corresponds with the amino acid of the related position in the first group of positions of the protein to said amino acid of the protein.

28. The method of claim 27 wherein, following the act of applying, the method further comprising:
shifting the template by 1 position along the protein in a selected direction to thereby define a second group of positions in the protein; and
repeating the act of applying the score with respect to the second group.

29. The method of claim 28 wherein the acts of shifting the template and repeating the act of applying the score are repeated until a desired region of the protein has been scored.

30. The method of claim 29 wherein the act of summing amino acid scores comprises summing said scores at each of a plurality of groups of positions of the protein.

31. The method of claim 30 wherein the act of comparing the stimulatory potency scores comprises comparing said scores for each of the plurality of groups of positions of the protein comprising the group having a desired level of stimulatory potency.

32. A method for determining the amino acid sequence of a peptide that stimulates a T cell, comprising:
providing a panel of peptides;
scoring the ability of each amino acid within said panel of peptides to stimulate a T cell; and
determining the amino acid sequence of the peptide that most effectively stimulates said T cell.

33. The method of claim 32, wherein said panel of peptides comprises a plurality of ten amino acid long peptides, wherein the amino acid sequence of the peptides are different from one another.

34. The method of claim 32, wherein scoring the ability of each amino acid to stimulate a T cell comprises determination of a positional scoring data on each amino acid.

35. The method of claim 34, wherein said positional scoring data is determined by a positional scoring matrix.

36. The method of claim 32, wherein determining the amino acid sequence of the peptide that most effectively stimulates said T cell comprises inputting positional scoring data into an artificial neural network (ANN).

37. A method for determining a protein that stimulates a T cell, comprising:
providing a plurality of peptides, wherein each peptide comprises a different amino acid sequence;
measuring the stimulatory potential of each peptide in said plurality of peptides for its ability to stimulate said T cell;
determining a first peptide in said plurality of peptides that most effectively stimulates said T cell; and
searching a database of protein sequences to identify a protein having the amino acid sequence of said first peptide.

38. The method of claim 37, wherein said plurality of peptides comprises subgroups of peptides, wherein the peptides in each subgroup have at least one of the same amino acid at the same position.

39. The method of claim 37, wherein peptides comprise ten amino acids.

40. The method of claim 37, wherein determining which peptide in said plurality of peptides comprises inputting measurements of T cell stimulatory data into an artificial neural network.

41. The method of claim 40, wherein said artificial neural network has been trained with said positional scoring data to determine an epitope which most strongly stimulates said T cell.


SEQUENCE LISTING (50 sequences)

SEQ ID NO: 1 - protein, 10 aa, Influenza virus
  PEPTIDE (308)...(317)
YVKQNTLKLA

SEQ ID NO: 2 - protein, 10 aa, H. sapiens
  PEPTIDE (89)...(98)
FFKNIVTPRT

SEQ ID NO: 3 - protein, 13 aa, Influenza Virus
  PEPTIDE (306)...(318)
PKYVKQNTLKLAT

SEQ ID NO: 4 - protein, 13 aa, H. sapiens
  PEPTIDE (87)...(99)
VHFFKNIVTPRTP

SEQ ID NO: 5 - protein, 87 aa, Unknown
  Peptide search supermotif
WYFRHMLIVADFYHKQVILYHKPTMNHQMTSNIQGVAHMGPAHFSTYVNQLICMRKGPMTNVSFRMYKLVHQPNISWGALMIFVYQA

SEQ ID NO: 6 - protein, 67 aa, Unknown
  peptide search supermotif
WYFRMLIVADFKQVILYHKPNHQTSNIQGVAHGPAHFSTYVNQLRKGPMFRMYKLVHQPNILMIFVY

SEQ ID NO: 7 - protein, 121 aa, Unknown
  peptide search supermotif
WHYFARTLCGQVKNKIFSRYLWMTAVNKDLCGFVIYQNHLKIMVSATDGVMLIWYTRVMILPTYSKWGEQNATSFVRWLQKGAPNYKICTSPLFQMRAHWFKRVPYLIHTISVHWKMAFLR

SEQ ID NO: 8 - protein, 66 aa, Unknown
  peptide search supermotif
WHYFARTLCGKIFSRYLKDLCLKIMVVMLIWVMILPTYSKTSFVRWKICTSPLFKRVPYLTISVHW

SEQ ID NO: 9 - protein, 10 aa, Artificial Sequence
  optimal theoretical peptide
WMKQNIGRFL

SEQ ID NO: 10 - protein, 10 aa, B. burgdorferi
NNIYKKALIS

SEQ ID NO: 11 - protein, 10 aa, B. burgdorferi
SNIIKSLSLF

SEQ ID NO: 12 - protein, 10 aa, B. burgdorferi
SNIIKKTSED

SEQ ID NO: 13 - protein, 10 aa, B. burgdorferi
FNIYKRVVDN

SEQ ID NO: 14 - protein, 10 aa, B. burgdorferi
NNIDKKVYTN

SEQ ID NO: 15 - protein, 10 aa, B. burgdorferi
FFIKKRSLII

SEQ ID NO: 16 - protein, 10 aa, B. burgdorferi
RNIFKKTVEN

SEQ ID NO: 17 - protein, 10 aa, B. burgdorferi
SNIKSKLILV

SEQ ID NO: 18 - protein, 10 aa, B. burgdorferi
YNIIVSSLLL

SEQ ID NO: 19 - protein, 10 aa, B. burgdorferi
DNIFKKETLI

SEQ ID NO: 20 - protein, 10 aa, B. burgdorferi
QAIGKKIQNN

SEQ ID NO: 21 - protein, 10 aa, B. burgdorferi
TLITKKISAI

SEQ ID NO: 22 - protein, 10 aa, B. burgdorferi
LNIKNSKLEI

SEQ ID NO: 23 - protein, 10 aa, B. burgdorferi
FNIIKVHSSL

SEQ ID NO: 24 - protein, 10 aa, B. burgdorferi
YNIKKIKVED

SEQ ID NO: 25 - protein, 10 aa, B. burgdorferi
LNITSSSYLF

SEQ ID NO: 26 - protein, 10 aa, B. burgdorferi
ENIKKILLRE

SEQ ID NO: 27 - protein, 10 aa, B. burgdorferi
NNIKSKVDNA

SEQ ID NO: 28 - protein, 10 aa, H. sapiens
YSICKSGCFY

SEQ ID NO: 29 - protein, 10 aa, H. sapiens
LHIISKRVEA

SEQ ID NO: 30 - protein, 10 aa, H. sapiens
SFIYSVVCLV

SEQ ID NO: 31 - protein, 10 aa, H. sapiens
GHIKKKRVEA

SEQ ID NO: 32 - protein, 10 aa, H. sapiens
FNITSSTCEL

SEQ ID NO: 33 - protein, 10 aa, H. sapiens
ENVKKSRRLI

SEQ ID NO: 34 - protein, 10 aa, H. sapiens
DNITSSVLFN

SEQ ID NO: 35 - protein, 10 aa, Human herpesvirus
FNIIKSLLGG

SEQ ID NO: 36 - protein, 10 aa, Human adenovirus
PNITFSVVYN

SEQ ID NO: 37 - protein, 10 aa, HIV
FNITSSIRNK

SEQ ID NO: 38 - protein, 10 aa, Human herpesvirus
ENIYYSSVRT

SEQ ID NO: 39 - protein, 6 aa, H. sapiens
YGGFMR

SEQ ID NO: 40 - protein, 6 aa, H. sapiens
YGGFLR

SEQ ID NO: 41 - protein, 6 aa, H. sapiens
YGGFMK

SEQ ID NO: 42 - protein, 6 aa, H. sapiens
YGGFLK

SEQ ID NO: 43 - protein, 6 aa, H. sapiens
YGGFMT

SEQ ID NO: 44 - protein, 10 aa, Influenza virus
YIKQNTLKLS

SEQ ID NO: 45 - protein, 10 aa, H. sapiens
  PEPTIDE (246)...(255)
YIDDNSKKVF

SEQ ID NO: 46 - protein, 10 aa, Artificial Sequence
  negative theoretical peptide
EPASAKEWDR

SEQ ID NO: 47 - protein, 8 aa, H. sapiens
SLYFCASS

SEQ ID NO: 48 - protein, 7 aa, H. sapiens
MPGQGGG

SEQ ID NO: 49 - protein, 15 aa, H. sapiens
TDTQYFGPGTRLTVL

SEQ ID NO: 50 - protein, 4 aa, H. sapiens
EDLK